(19) United States
(12) Patent Application Publication
(10) Pub. No.: US 2006/0265077 A1
(43) Pub. Date: Nov. 23, 2006

(54) Spinal repair

(76) Inventor: Paul A. Zwirkoski, Pinckney, MI (US)

Correspondence Address: MORRISON & FOERSTER LLP, 755 PAGE MILL RD, PALO ALTO, CA, 94304-1018, US

(21) Appl. No.: 11/298,138

(22) Filed: Dec. 9, 2005

Related U.S. Application Data
(60) Provisional application No. 60/655,829, filed on Feb. 23, 2005.
(60) Provisional application No. 60/697,291, filed on Jul. 6, 2005.
(60) Provisional application No. 60/714,677, filed on Sep. 7, 2005.

(51) Int. Cl. A61F 2/44

(52) U.S. Cl. 623/17.16; 623/17.11; 623/17.14

(57) ABSTRACT

A spinal implant for repairing a region of a subject's spine may have a plurality of interlockable segments that can be deployed from a delivery configuration (e.g., a linear array) into a deployed configuration. When the implant is in the delivery configuration, the implant comprises a linear array of the segments that are flexibly connected, and when the implant is in the deployed configuration, the segments are interlocked into a stable structure so that each segment is adjacent to and interlocked with at least two other segments. in the deployed configuration, the implants may have a greater strength (e.g., crush strength) and may help maintain the stability of the body region. The implant may be inserted into the spinal region by an applicator from the posterior region of the subject in the delivery configuration and assembled within the body to form the deployed configuration.

### CROSS-REFERENCE TO RELATED APPLICATIONS

The present invention is related to and claims the benefit of U.S. Provisional Patent Application Ser. No. 60/655,829, filed Feb. 23, 2005, titled “METHODS AND APPARATUSES FOR FILLING A CAVITY II,” U.S. Provisional Patent Application Ser. No. 60/697,291, filed Jul. 6, 2005, titled “SPINAL REPAIR,” U.S. Provisional Patent Application Ser. No. 60/714,677, filed Sep. 7, 2005, titled “NON-SOFT TISSUE REPAIR,” and U.S. Provisional Patent Application Ser. No. ______ (not yet assigned), filed Nov. 23, 2005, titled “NON-SOFT TISSUE REPAIR II,” by Paul Zwirkoski, the disclosures of which are herein incorporated by reference in their entirety.

### FIELD

Described here are spinal implants, implant applicators, delivery devices, and methods for using them. In particular, the description relates to implants having a plurality of interlockable, flexibly connected segments that may individually or once assembled have a strength sufficient to support, to fill, to create, to maintain, to distract, or to otherwise repair a portion of the spine, including bone cavities, the intravertebral region of a spine, and the intervertebral region of a spine.

### BACKGROUND

Proper treatment of spinal injuries such as trauma, fractures, non-unions, tumors, cysts, and degenerated discs may involve filling a cavity that has been created by the pathology itself or by the action of a surgeon. Often the cavities are compressed, and require that the surfaces of the cavity be distracted from one another and then supported to return the spinal structure to its anatomic position and form. Furthermore, because spinal tissues such as vertebra and cartilage have structural and support roles in the body, it is critical that such cavities be repaired to allow reliable strength and support.

Compression fractures are one type of hard tissue injury belonging to a class of conditions that may be treated using devices and methods for separating, distracting, and supporting a fractured bone. For example, vertebral compression fractures are crushing injuries to one or more vertebra. A vertebral compression injury may be the result of a trauma to the spine, an underlying medical condition, or a combination of a trauma and an underlying condition. Osteoporosis and metastatic cancers are common medical conditions that also contribute to vertebral compression fractures because they weaken spinal bone, predisposing it to compressive injury.

Osteoporosis is a degenerative disease that reduces bone density, and makes bone more prone to fractures such as compression fractures. An osteoporosis-weakened bone can collapse during even normal activity. According to the National Institute of Health, vertebral compression fractures are the most common type of osteoporotic fractures.

Vertebral fractures may be painful and may deform the shape of the spine, resulting in unhealthy pressure on other parts of the body, loss of height, and changes in the body's center of gravity. Untreated, such changes and the resulting discomfort can become permanent, since the bone heals without expanding the compression.

Existing methods of treating bone injuries may involve highly invasive or inadequate treatments. For example, one method of treatment is percutaneous vertebroplasty. Vertebroplasty involves injecting bone filler (such as bone cement) into the collapsed vertebra to stabilize and strengthen the crushed bone. In vertebroplasty, physicians typically insert a small diameter guide wire or needle along the pedicle path intended for the bone filler delivery needle. The guide wire is advanced into the vertebral body under fluoroscopic guidance to the delivery point within the vertebrae. The access channel into the vertebra may be enlarged to accommodate the delivery tube. In some cases, the delivery tube is placed directly into a vertebral body and forms its own opening. In other cases, an access cannula is placed over the guide wire and advanced into the vertebral body. In both cases, a hollow needle or similar tube is placed into the vertebral body and used to deliver the bone filler into the vertebra.

When filling a bone cavity with bone filler using traditional vertebroplasty, fillers with lower viscosities may leak. Further, even fillers having low viscosities may require the application of a high pressure to disperse the bone filler throughout the vertebral body. However, application of high pressure also increases the risk of bone filler extravasation from the vertebral body. Conversely, injecting a bone filler having a higher viscosity may provide an even greater risk of “leaking” bone filler into sensitive adjacent body areas. Leaks or extrusion of the bone filler may be dangerous to a patient's health. For example, posterior extravasation from a vertebral body may cause spinal cord trauma, perhaps resulting in paralysis. Risk of leakage is even more acute when a bone filler is applied under pressure to expand a compression fracture, especially if the fracture has begun healing and requires substantial force to distract the cavity surfaces.

Furthermore, most bone cements and bone fillers are difficult to remove or to adjust. Removal and adjustment may be important when distracting a bone cavity. For example, removing a precise amount of bone filler may allow a surgeon to adjust the level of distraction of a vertebral compression fracture and correct the shape of the compressed bone. Many bone cements, once set, are difficult or impossible to remove without further, highly invasive, surgery. Even if the removal is attempted prior to the expiration of the setting time, the materials may have non-Newtonian flow characteristics requiring a substantial removal vacuum to achieve an initial and sudden movement.

The implants described herein may avoid many of the problems described above when filling a cavity within the body, and particularly a cavity within the spinal region. The use of segments contained within a flexible tube or sheath offers an alternative to packing or expanding a cavity within body tissue. This could be an advantage in the treatment cavities such as vertebral compression fractures since the use of a flexible tube reduces concerns of fluent material leakage from the internal vertebral space and provides more control in delivery. These devices may be used in other regions of the body where the filling of a cavity with stability and control is desired, and is not necessarily limited to the spinal region. For example, the devices described herein may be used to repair hip, tibia, and other areas of bone displacement.

In addition to traditional bone cements, a handful of other cavity filling materials have been suggested. In particular, biodegradable and/or bioabsorbable devices have been suggested. For example, U.S. Pat. No. 5,756,127 to Grisoni et al. describes a bioresorbable string of calcium sulfate hemihydrate (Plaster of Paris) beads and a means for producing these beads. However, the Grisoni device is not appropriate for spinal regions, and has many disadvantages. Calcium sulfate hemihydrate (Plaster of Paris) and similar materials have low elasticity and crush strength, making them unreliable as materials to distract and later support a spinal region, particularly during the early stages of the healing process. Filling materials that are readily compressed or crushed may shift within, or exit, the cavity altogether, leading to detrimental changes in the shape of the spinal region. Materials with low crush strength are poor choices in withstanding the stress of distracting spinal regions, and may be unable to maintain the distracted shape after filling a spinal region. Similar materials are the subjects of U.S. Pat. No. 6,579,533 to Tormala et al.

U.S. Pat. No. 5,702,454 to Baumgartner describes an implant made of an elastic plastic for implanting into an intervertebral disk. Because the Baumgartner implant is elastic and somewhat amorphic, it may be less effective for filling and distracting spinal cavities, particularly cavities benefiting from implants having some stiffness, such as non-soft tissue cavities, and cavities that benefit from a stable implant shape. This is particularly true where sustained distraction is desired.

U.S. Pat. No. 6,595,998 to Johnson et al. describes a tissue distraction device in which wafers are inserted to distract a tissue cavity by forming a wafer stack within the cavity. However, Johnson's column of wafers is not amenable to providing uniform support to all surfaces of a cavity, when such support is needed. For example, a tissue cavity supported or distracted on all sides of the cavity may be more stable.

U.S. Pat. No. 5,958,465 to Klemm et al. describes a method and apparatus for making drug-containing implants in the form of a string of beads comprising chains of small drug-containing plastic bodies arranged in series on a surgical wire or thread. Similar drug implanted beads-on-a-string are described in U.S. Pat. No. 6,183,768 to Harle and German Patents 2320373 to Klemm and 2651441 to Heusser. The Klemm, Harle, and Heusser implants are designed for drug delivery, and are embedded with one or more drugs which are released from the plastic (e.g. PMMA) beads (also called “corpuscles”). Thus, these implants may be limited in strength and durability because of the inclusion of a releasable drug, as well as the properties and shape of the implant beads.

In any event, none of the cited documents show the device and methods disclosed below. The devices described herein may address many of the problems identified above, particularly in the treatment of the spine.

For example, the devices methods and systems described herein may be particularly useful as interbody implants for the treatment of spinal regions. Interbody implants may include interbody fusion devices, replacement discs or nuclear replacement. The tensioned, segmented and interlocking devices described herein may be particularly useful as interbody implants for treating the spinal region.

Interbody implants offer an alternative to the current practice of fusing vertebra (e.g., using pedicle screws and/or hook constructs in conjunction with an interveterbral body cage and/or bone graft or other intervertebral techniques). Current techniques and devices for spinal repair may be unsuccessful due to less bony endplate coverage, which translates into less load transfer and may result in loosening, shifting, and other failures of the treatment. Segmented implants, as described herein, may be used for interbody implants, including interbody fusion implants, nucleus replacement, total disc replacement, or any other interbody implant.

Successful interbody implants have been difficult for several reasons. For example, a significant challenge in performing a successful interbody implant surgery is the small size of the entry portal providing access to the intervertebral region. Achieving a long term final stabilization of the implant has also been a challenge. Existing devices are typically assembled prior to delivery into the intervetebral space, or are only a single (large) peice. Their size requires large portal entries and, in many cases, must be delivered anteriorly. This is particularly true with traditional artificial disc replacements. In contrast, the implants described herein may be assembled within the intervetebral space after delivery through a portal, substantially reducing the portal opening size needed.

Spinal interbody fusion (IBF) surgical intervention is intended to limit or stop motion between two vertebral levels. It also is secondarily utilized to restore the height of the interbody disk space. One indication for IBF is the unsuccessful relief of back pain after less invasive alternative therapies fail. Pain relief can be achieved with IBF by relieving the inflammation of neural structures caused by motion at the disk level. This motion causes pain because the vertebral bodies irritate the adjacent neural structures by loading, constricting and/or abrading them, and is typically part of the progression of Disk Degenerative Disease (DDD). Typical methods of performing IBF place one or two small curved spacers that can distract and fuse the adjacent vertebral bodies into the intervertebral space. The spacers are designed to allow bone growth and consolidation, and ideally remain in a fixed location affording bony ingrowth that will result in long term fixation. However, these spacers can shift, leading to fusion failure. Other devices have similar problems since none form a structure that cannot be independently shifted, such as a stable ring or circle. As described more fully below, implants comprising interlocking and connected segments may be beneficially used as interbody implants.

Nucleus replacement is another type of spinal repair that may benefit from segmented implants as described herein. Nucleus replacement is intended to restore motion between two vertebral levels. For example, nucleus replacement may include replacing all or most of the disc tissue by implanting a replacement device into the space between the vertebrae. In interbody disc nucleus replacement, only the center of the disc (the nucleus) is removed and replaced with an implant. The outer part of the disc (the annulus) is not removed. Typical hydrogel replacements (e.g., commonly used for nucleus replacements) are large and thus require large entry ports. Further, they also have a high failure rate due to implant slippage and/or displacement. Thus, most existing devices or implants used for nucleus replacement are limited by the large portal dimensions required for implantation of the device, and also have a tendency to be quite unstable.

Total disc replacement (also known as disc artheroscopy) replaces a damaged disc to restore the height, motion, and flexibly to a region of the spine. Typically, total disc replacement requires insertion of one or more large plate-like implants between two vertebrae after removing the disc. In some typical operations, two or more layers are inserted. For example, each layer may be a plate that is attached to the upper or lower vertebra. A third layer may be inserted between these two plates. In order to accommodate the implant(s), a large access portal must be cut into the subject, resulting in pain, an extended recovery time, and damage to otherwise healthy tissue. It would be beneficial to provide a total disc replacement device and method that does not require a large portal size, as well as a total disc replacement device that may be readily adjusted by the surgeon during insertion.

### BRIEF SUMMARY

Broadly, described here are segmented implants for filling a tissue cavity, applicators for inserting implants, and methods of using the segmented implants and applicators to fill and/or distract tissue cavities. In particular, the implants described here may be used for filling and/or distracting non-soft tissue cavities such as a bone cavity, and for anchoring devices (e.g., bone screws, etc.) within the body. Generally, the segmented implants described here comprise a plurality of segments that interlock to form an assembled structure

Described herein are implants for insertion into the spinal region of a subject, comprising a plurality of interlockable segments that are deployable from a delivery configuration into a deployed configuration. When the implant is in the delivery configuration, the implant comprises an array (e.g., a linear array) the segments that are flexibly connected. When the implant is in the deployed configuration, the segments are interlocked into a stable structure so that each segment is adjacent to and interlocked with at least two other segments. The deployed configuration (which may also be referred to as an “assembled” configuration) may have a greater strength and/or stability than the individual segments, or even an aggregate of segments. A filament may also connect the segments of the implant, and at least some of the segments are slideably coupled to the filament.

The segments of the implant may interlock by mating with adjacent segments in the deployed configuration. For example, some (or all) of the segments may include cavities configured to mate with teeth on other segments. Interlocking may be temporary or permanent. In some variations, a holdfast may be used to secure the implant in the deployed (and interlocked) configuration. A single holdfast may be used for the entire implant, or multiple holdfasts may be used (e.g., between each or a subset of segments). Any appropriate holdfast may be used, including adhesives, mechanical fasteners, electrical/magnetic fasteners, etc.

The implants may have any appropriate deployed configuration, particularly deployed configurations that are stable. Particularly stable configurations include deployed configurations in which each segment is adjacent (and interlocked) with at least two other segments. For example, the implant may have a deployed configuration that is a ring, a disc, a sphere, etc. Thus, individual segments may be configured so that they assemble to form the correct deployed configuration. For example, when the deployed configuration is a disk, at least some of the segments may be substantially pie-shaped. At least some of the segments may comprise tissue-engagement surfaces configured to stabilize the implant in the tissue when the implant is in the deployed configuration.

In any of the variations of the implant, the implant may include an orientation guide configured to maintain the orientation of the segments with respect to each other in the delivery configuration. Orientation guides may be helpful in orienting the segments so that they can readily assemble into the deployed configuration. The orientation guide may be grooves, tethers, joints, or the like. For example, the orientation guide may comprise a filament connecting the segments.

Also described herein is a method of inserting an interlockable implant. The method may include positioning an applicator adjacent to a target tissue site, delivering an implant to the target tissue site (wherein the implant comprises a linear array of flexibly connected interlockable segments), and securing the implant into a deployed configuration (wherein the interlockable segments are interlocked so that each segment is adjacent to and interlocked with two other segments).

The method may also include the step of deploying the implant from a delivery configuration in which the implant comprises a linear array of flexibly connected segments to a deployed configuration in which the segments are interlocked into a stable structure so that the interlocked segments do not move with respect to adjacent segments. The step of deploying may include tensioning the connection between the segments.

In some variations, the implant is configured as a ring-shaped interbody fusion device for inserting into the spinal region of a subject in need thereof. For example, an interbody fusion device for insertion into the spinal region of a subject may include a plurality of interlockable segments that are deployable from a delivery configuration into a deployed configuration. When the implant is in the delivery configuration, the implant comprises a linear array of the segments that are flexibly connected. When the implant is in the deployed configuration, the segments are interlocked into a ring wherein each segment is adjacent to and interlocked with at least two other segments. At least some of the segments may comprise voids configured to allow ingrowth of tissue.

The segments of the interbody fusion device may have two faces that are offset by between about 30 and about 60 degrees, wherein each face is configured to interlock with an adjacent face of a another segment.

A method of inserting an interbody fusion device may include the steps of positioning an applicator adjacent to a target tissue site, delivering an interbody fusion device to the target tissue site (wherein the device comprises a linear array of flexibly connected interlockable segments), and securing the device into a deployed configuration wherein the interlockable segments are interlocked so that each segment is adjacent to and interlocked with two other segments to form a ring.

In some variations, the implant is configured as a nuclear replacement device for insertion into a subject's spine. Nuclear replacements may be substantially disc-shaped. The nuclear replacement insert may include a plurality of pie-shaped, interlockable segments that are deployable from a delivery configuration into a deployed configuration. When the implant is in the delivery configuration, the implant comprises a linear array of the segments that are flexibly connected. When the implant is in the deployed configuration, the segments are interlocked into a disc wherein each segment is adjacent to and interlocked with at least two other segments. At least a region of the segments of the nuclear replacement device may comprise an elastic material (including a coating, etc.). The segments of the nuclear replacement device may have two faces that are offset by between about 30 and about 60 degrees. Each face may be configured to interlock with an adjacent face of a another segment.

A method of inserting a nuclear replacement device may include positioning an applicator adjacent to a target tissue site, delivering a nuclear replacement device to the target tissue site (wherein the device comprises a linear array of flexibly connected interlockable segments), and securing the device into a deployed configuration wherein the interlockable segments are interlocked so that each segment is adjacent to and interlocked with two other segments to form a disc.

In some variations, the implant is configured as a total disc replacement assembly or device for insertion into a subject's spine. The total disc replacement device may include a plurality of interconnecting segments, deployable from a delivery configuration into a deployed configuration. The segments may include a central endplate and a plurality of wing segments. When the implant is in the delivery configuration, the implant may comprise a central endplate and a linear array of the wing segments that are flexibly connected. When the implant is in the deployed configuration, the segments may be interlocked around the central endplate into an articulating endplate.

The central inner bead may be configured to abut a second articulating endplate. In addition, the central endplate may comprise a disc having a channel for mating with the wing segments. At least a region of the central endplate may comprise a smooth surface for mating with a central inner bead. In some variations, the annular endplate comprises a concave surface for coupling with the central inner bead.

The total disc replacement device may also include a central inner bead configured to abut at least the central endplate region of the articulating endplate. In some variations, the total disc replacement device includes a second plurality of segments comprising a second central endplate and a second plurality of wing segments. When the device is in the delivery configuration, the device comprises a second central endplate and a linear array of the second wing segments that are flexibly connected, and when the device is in the deployed configuration, the second plurality of segments are interlocked around the second central endplate into a second articulating endplate.

Also described herein is a method of inserting a total disc replacement device. The method may include positioning an applicator adjacent to a target tissue site, delivering an articulating endplate to the target tissue site (wherein the articulating endplate comprises a linear array of flexibly connected interlockable segments comprising a plurality of wing segments for mating with a central endplate), and securing the articulating endplate into a deployed configuration wherein the wing segments and the central endplate are interlocked. The method may also include a step of delivering a central inner bead to the target tissue site. In some variations, the method also includes the step of delivering a second articulating endplate to the target tissue site, wherein the second articulating endplate comprises a linear array of flexibly connected interlockable segments comprising a plurality of wing segments for mating with a central endplate.

### BRIEF DESCRIPTION OF THE DRAWINGS

Embodiments or variations are now described by way of example with reference to the accompanying drawings.

FIGS. 1A to 1E show variations of the described implant;

FIGS. 2A to 2F show variations of the described implant;

FIGS. 3A to 3E, 3G, 3I to 3N, 3P to 3T show variations of the described implant;

FIGS. 3F, 3H, 3W and 3X illustrate variations of interlocking segments of the described implant;

FIGS. 4A to 4D show variations of the described implant;

FIG. 5 illustrates a variation of an applicator for the implant;

FIGS. 6A to 6C illustrate variations of the distal cannula tip of an applicator;

FIGS. 7A and 7B show one variation of an applicator driver;

FIG. 7C shows another variation of an applicator driver;

FIG. 7D shows the relationship between an applicator and variations of the driver;

FIGS. 8A to 8C show insertion of an implant into a vertebral body;

FIGS. 9A and 9B show a screw closure compatible with the implants and applicators described herein. FIG. 9B is a schematic cross-section of the screw closure shown in FIG. 9A taken along the longitudinal plane A-A.

FIG. 10 shows a cutter for cutting segments of the implant as described herein.

FIGS. 11A to 11I shows exemplary implants as described herein.

FIGS. 12A to 12D illustrate insertion of the implants as shown in FIG. 11 into a disk (e.g., nucleus disk) region.

FIGS. 13A and 13b shows an applicator that may be used with the implants shown in FIG. 11.

FIGS. 14A to 14C show perspective views of one various segments as described herein.

FIGS. 15A and 15B show a top and a perspective view of a segment as described herein.

FIGS. 16A to 16F show top, side, cross-sectional and perspective views of different segments as described herein.

FIGS. 17A to 17F illustrate assembly of an implant as described herein.

FIGS. 18A to 18C illustrate variations of segments.

FIG. 19 illustrates an implant that may be suitable for total disc replacement as described herein.

FIGS. 20A and 20B show cross-sectional views of an implant as shown in FIG. 19.

FIGS. 21A to 21C show segments of an implant as shown in FIG. 19.

FIGS. 22A to 22D show side, cross-sectional, top and perspective views, respectively of a segment, as described herein.

FIGS. 23A to 23D show variations of a CIB segment, as described herein.

FIGS. 24A to 24D show top and perspective views of another variation of a segment.

FIGS. 25A to 25D show perspective views illustrating assembly of one variation of an implant.

FIG. 26 shows a perspective view of one region of an implant being assembled, as described herein.

FIGS. 27A and 27B show top views of one portion of an implant as described herein.

FIG. 28 shows a hybrid ram applicator as described herein.

FIGS. 29A to 29D show components of the hybrid ram applicator of FIG. 28.

FIGS. 30A to 30C show cross-sections through the hybrid ram applicator of FIG. 28.

FIGS. 31A to 31C show perspective views of the internal cannula region of the hybrid ram applicator of FIG. 28.

FIGS. 32A to 32B show perspective views of the reciprocating ram region of the hybrid ram applicator of FIG. 28.

FIGS. 33A to 33C show perspective and cross-sectional views of the outer sheath region of the hybrid ram applicator of FIG. 28.

FIGS. 34A, 34B, and 34C show perspective views of the flexibly connected segments within flexible tubes instruments and implants described herein.

FIGS. 35A and 35B show perspective views of segments with sharp protusions contained within the flexible tube instrument and implant of FIG. 34B.

FIGS. 36A, 36B and 36C show perspective views of crushable and pervious segments within the flexible implant of FIG. 34B.

FIG. 37 show a perspective view of single chains of segments at the top and bottom of a cavity, where the space between the chains contains flexible implants of FIGS. 34A and 34B.

FIG. 37B shows a perspective of a vertebral cavity that contains a UV hardened settable material.

FIG. 38 shows a locking device with an implant configured as an anchor.

FIG. 39 shows a cross-section of one variation of a total disc replacement assembly including a centering structure.

FIG. 40 shows another variation of a total disc replacement assembly including a centering structure.

FIG. 41 shows a total disc replacement assembly which includes a circumferential compliance ring.

FIGS. 42A and 42B illustrate another variation of an articulating endplate.

### DETAILED DESCRIPTION

In the drawings, reference numeral 10 generally denotes an exemplary embodiment of a segmented implant for distracting, filling, creating, or maintaining a cavity in a tissue. The implant, applicator, and methods of use may be used for distracting, supporting, filling, creating and maintaining the size of virtually any tissue cavity, particularly hard tissue cavities, including but not limited to: bone separations, fractures (including compression fractures), non-unions, removed tumors, removed cysts, in conjunction with joint replacement implants, and certain fusion procedures. Although example of implants, implant applicators, combinations of implants and applicators and methods of using the implants are described in the context of treating a vertebral compression fracture, the devices and methods of use described are not intended to be limited to vertebral compression fractures.

The implants, applicators and methods described herein are particularly relevant to insertion into body regions such as non-soft tissue cavities. Non-soft tissue cavities include hard tissues cavities such as cavities or voids such as bones, as well as cartilage, and bone connected to ligament and/or muscle, scar tissues, and other mineralized (e.g. calcified) tissues. Non-soft tissue cavities also include tissues cavities having at least one hard surface, including tissues having mixed compositions. For example, non-soft tissue cavities include cavities abutting bone, or cavities surrounded by bone, such as cavities within the spinal disk space, cavities within the bone marrow, and cavities adjacent to bone or bone and ligament.

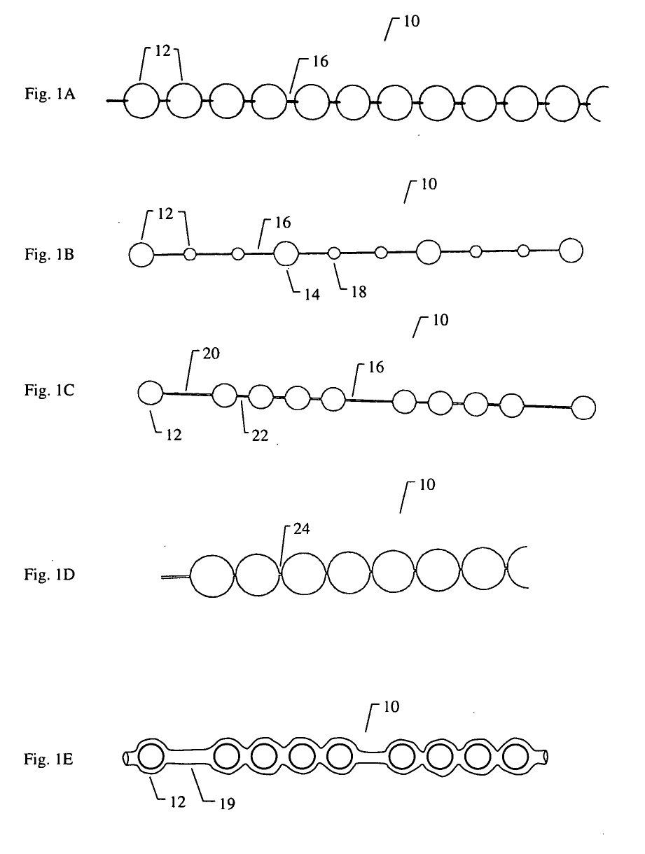

FIGS. 1A to 1E illustrate variations of implants for distracting or filling a tissue cavity. The implant 10 in each of FIGS. 1A to 1E includes a plurality of segments (illustrated as pellets) that are flexibly joined. Segments of the segmented implants may include one or more pellets. A perspective view of an implant is shown in FIG. 1A. The segments 12 are shown as spherical pellets that are connected by a centrally located wire, string, or fiber 16. The joined pellets form a connected construct seen as a flexible linear array that may be inserted into a cavity to distract the cavity walls, to fill the cavity, or to provide continuing support to the cavity. As used herein, unless the context makes clear otherwise, “distract” or “distracting” refers to the process of separating (or enlarging) the walls of a cavity, particularly a bone cavity.

Crush Strength

An implant may be used to distract, to fill, to create or to maintain the size or shape of a hard tissue body cavity such as a bone cavity. In one variation, the described implant's segments 12 have crush strength adequate to withstand the forces required to distract and support the cavity without substantial compression or breaking of the segments. Crush strength is defined as average crush load per unit cross-sectional area at which the structure will break or crack, and may be expressed in pounds per square inch or megaPascals (MPa). Of course, the shape of a segment has both individual and group effects upon the crush strength of the implant after installation. The crush strength of an individual segment pellet, however, is a consideration for distracting a cavity. For roughly spherical pellets, force can be approximated as acting at discrete points on the surface of the sphere, so crush force may be approximated as the total force applied to crack the sphere. One factor effecting crush strength is compressible strength of the material.

Compressibility

It may be beneficial that the segments comprise any solid material having an appropriate compressible strength so that the implant assemblage is able to distract, fill and support a tissue cavity without substantially deforming. The segments preferably comprise biocompatible solids with high compressive strength. Compressibility and incompressibility generally describe the ability of molecules in a solid to be compacted or compressed (made more dense) under an applied force and/or their ability to return to their original density after removing the applied force. Compressibility of a solid may also be quantified by the bulk modulus of the substance (bulk modulus is the inverse of compressibility, and is the change in volume of a solid substance as the pressure on it is changed). A relatively incompressible material will have a higher bulk modulus than a more compressible material.

The compressive strength of cortical bone is approximately 166 MPa, and the compressive strength of cancellous (spongy) bone is approximately 4 MPa. In one variation, the implant should have a compressive strength of greater than approximately 20 MPa. In one variation, the implant should have a compressive strength less than cortical bone. In one variation, the implant has a compressive strength between about 20 and about 160 MPa. In one variation, the implant has a compressive strength between about 91 and about 160 MPa. In one variation, the implant has a compressive strength between about 100 and about 160 MPa. As a reference, the compressive strength of calcium sulfate is approximately 11 MPa.

The implant or segments of the implant may also have a mixed compressibility or crush strength, because a portion of the implant may be more compressible than another portion of t the implant. For example, the implant may have a layer or coating of elastic or other compressible material. In some variations, the different segments may have different compressibilities.

Segment Materials

The crush strength of the implant depends to a large extent, on the segment crush strength, which is a function of the composition, and to a lesser degree, the shape of the segment.

Materials with appropriate crush strength include, but are not limited to, metals, alloys, ceramics, certain inorganic oxides and phosphates, polymers, bone derived material, and combinations of these materials. The following descriptions of segment materials represent variations of the implant, and are not intended to limit the scope of the implant or segment materials. The implant segment may comprise, consist of, or consist essentially of the materials identified herein.

Bioabsorbable (or bioerodible) and non-bioabsorbable (or non-bioerodible) material may be used in the implant separately or in combination. Typically, the non-absorbable (or non-bioerodible) materials noted elsewhere provide segments and implants exhibiting a sustainable crush strength adequate to maintain the distraction of the cavity surfaces (e.g. bone cavity surfaces) over a long period of time. On the other hand, bioabsorbable (or bioerodible) segments exhibit a reduction in crush strength over time, as the material is acted upon by the body. However, bioabsorbable materials may also permit substantial tissue in-growth, allowing tissue to replace implant material while maintaining the distraction and supporting the filled cavity. In applications in which the likelihood of tissue re-growth is small, for example osteoporotic repair, a nonabsorbable implant may be desirable. Materials that are too rapidly bioabsorbed (for example, calcium sulfate hemihydrate) are generally inappropriate as segment materials, because they do not maintain the cavity structure and/or distraction.

Metals that may be used as segment materials include, but are not limited to, biocompatible metals and alloys, such as stainless steels, gold, silver, tantalum, cobalt chromium, titanium, platinum, rhodium, rhenium, ruthenium, and other alloys thereof, combinations thereof, or other equivalent materials.

Ceramic materials that may be used in the segments may include, but are not limited to, alumina, carbon or tricalcium phosphate or sintered masses or single crystals of hydroxyapatite. Ceramics capable of high crush strengths may be particularly relevant. Also useful are refractory metal and semi-metal oxides (tantalum oxides, aluminum oxides), phosphates (calcium phosphates), phosphides, borides (niobium borides, tungsten borides), carbides (aluminum carbides, boron carbides, niobium carbides, silicon carbides, tantalum carbides, titanium carbides, tungsten carbides, vanadium carbides, zirconium carbides), nitrides (boron nitrides, chromium nitrides, silicon nitrides, tantalum nitrides, titanium nitrides, zirconium nitrides), silicides (tantalum silicides, tungsten silicides, zirconium silicides), their mixtures, variously sintered as porous particulates or as solid formations.

Inorganic materials that may be used as segment materials include, but are not limited to, hardened glasses including oxides of silicon, sodium, calcium and phosphorous and combinations thereof.

Polymers that may be used as segment materials include, but are not limited to, elastomers (natural and synthetic rubbers, silicone rubbers), polymethyl methacrylate (PMMA), polyetheretherketone (PEEK), polymethymethacrylate (PMMA), polyglycolic acid and/or polylactic acid compounds, polyvinylchloride (PVC), polyethylene (PE, HDPE, UHMWPE, etc.), polystyrene (PS), polyesters (PET, polycaprolacton, polyglycolied, poylactide, poly-p-dixanone, poly-hydroxy-butylate), polyamides (Nylons, aromatic polyamides), polypropylene (PP), fluorocarbon polymers (PTFE, PTFCE, PVF, FEP) and other biocompatible materials. Other suitable polymers include: collagen and/or collagen derivative preparations alone or in combination with other biomaterials, chitin and chitosan preparations.

Bone derived materials that may be used as segment materials include, but are not limited to, bone autografts, bone allografts, bone xenografts, bone-derived tissue, bone-derived collagen, and the like.

Any combinations of these materials may be used as a segment material. Segments may include pellets of any of these materials, or combinations thereof. Finally, suitable known materials acceptable for use as hard tissue implant materials include various osteogenic and osteoinductive compositions, and combinations thereof. Certain glassy carbon forms are also quite useful.

Segment materials may also comprise radiopaque materials to enhance visualization of the implant, or the segments may incorporate a radiopaque material as a part of a segment (e.g., coatings, dispersed, or core materials). Examples of radiopaque materials include but are not limited to, barium sulfate, tungsten, bismuth compounds, tantalum, zirconium, platinum, gold, silver, stainless steel, titanium, alloys thereof, combinations thereof, or other equivalent materials for use as radiographic agents.

Coatings

Segments may include coatings to modify the surface properties of the segments, to have a biological effect, and/or to facilitate the insertion or removal of the implant. The coatings may be of any thickness. In one variation, the segment comprises layers of materials. In one variation, the segment has a hollow core.

In one variation of the implant described herein, a segment or segments may be coated with a therapeutic or medicinal material, such as an antibiotic. Additional medicinal materials may include, but are not limited to, anticoagulants and bone-growth promoting agents. In one variation of the implant, the segments may be coated with a cross-linking or bonding compound that could facilitate adhesion either between the segments, with the body region, or both. In one variation the segments are coated with a cross-linker that can be activated after insertion into the bone cavity, for example, by adding an activating compound, by time delay, or by temperature. In one variation the segments are coated with a lubricant.

The segments may comprise one or more therapeutic or medicinal materials situated away from the surface, e.g., in pores within the segments.

Drug Delivery Using the Implant

The segments may also be embedded with one or more therapeutic or medicinal materials. For example, embedding the segments with an additional material may be particularly useful when the segment comprises a bioabsorbable (bioerodible) material. Thus, the segments may be used to deliver any drug or therapy. Drugs which are particularly useful may include, but are not limited to, growth factors and/or growth promoters (e.g. bone derived growth factors (BDGF), bone morphogenetic protein (BMP), etc.), antibacterials, antivirals, vascularizing agents, analgesics, anticoagulants, cell and/or gene therapies, etc.

In one variation an implant including a drug is inserted at or near a wound site. After an appropriate time the implant is removed. Thus, the implant may serve as a removable wound packing material. In one variation, the implant may be inserted with a removable drain. In one variation, the implant functions as a removable drain.

Any portion of the implant may be coated with, implanted with, embedded with, or made from a therapeutic or medicinal material, including but not limited to those described herein.

Flexible Joining Material

The implant segments may be connected in the implant as it is installed. The segments may be linked together in such a way that each segment in the implant is adjacent, perhaps directly adjacent or in contact with at least one other segment. Generally, each segment in the implant is adjacent, perhaps directly adjacent or in contact with at most two other segments. In some variations, the assembled segments form a linear array. In the variation of the implant shown in FIGS. 1A to 1E, the segments are linked in a linear array by attachment to a wire, filament, or string 16. The filament connecting the segments may comprise a separate, independent filament between each segment, or it may be a single continuous filament. The filament may comprise different materials, and may be different lengths. In one variation of the implant, the filament comprises one or more monofilaments. In another variation of the implant, the filament comprises one or more fibers. In a variation of the implant, the filament comprises one or more wires. The filament may comprise a bioabsorbable material. The filament may be rapidly bioabsorbable because (unlike the segments) the filament is not typically load bearing in supporting the cavity.

In one variation, the implant segments are connected in any way allowing sufficient flexibility to the resulting implant construct so that it may be introduced into a cavity such as a bone hollow. In one variation, the implant segments are flexibly connected so that a segment may contact another segment upon being implanted into a body region such as a bone hollow.

The connection material may comprise, for instance, a string, fiber or wire, variously of single or multiple strands. The connecting string or fiber may be flexible and allow the segments to be inserted into the treatment site. Suitable filament materials include virtually any biocompatible material, including but not limited to: natural materials (e.g. cottons, silks, collagen, etc), rubbers (e.g. natural and synthetic rubbers), composite yarns (e.g. carbon fiber yarns, ceramic fibers, metallic fibers), polymers (e.g. polyethylene, polyester, polyolefine, polyethylene terephthalate, polytetrafluoroethylene, polysulfone, nylons, polylactic acids, polyglycolic acids, mixtures and copolymers of polylactic and polyglycolic acids (PGLA such as “Vicryl” from Ethicon and “Dexon” from Davis & Geck), polydioxanone, various Nylons, polypropylene, etc., and the like). Suture material (natural and synthetic materials) are examples of particularly appropriate materials.

In one variation, the segments are adapted to connect to the filament, string or wire, for example, by having holes (through which the flexible joining material is threaded), by having attachment sites (to which the flexible joining material could be tied or otherwise attached), or by having a track or groove (which mate to the flexible joining material). In one variation the segments are adherent to the string or filament by a glue, adhesive, or the like.

In one variation, the segments are connected by adhesives or glues, such as solvent- or catalyst-curable materials including Silicone glues, rubbery epoxies, and adhesives suitable for the materials forming the segments. In one variation the segments are connected only by adhesives or glues such as those mentioned above.

The joining material does not itself have to be flexible, so long as it allows flexibly joined segments of an implant to “flex.” In one variation of the implant, the segments are linked together by a solid linker. The implant is made flexible by incorporating a joint (e.g. socket type joins) between the solid linker and the segment. Solid linkers may be composed of the same material as the segments. Solid linkers may be wires made of one or more filaments comprising suitably biocompatible metals or alloys, e.g., stainless steels or superelastic alloys.

The flexible joining material may comprise any suitable materials including but not limited to: polymers, (e.g., polyfluorocarbons such as the various Teflons (including PTFE and expanded PTFE—ePTFE such as is sold as GORETEX), polypropylene, polyethylene, polyoxymethylene, polycarbonate, polyesters (including polyamides such as the Nylons), polyphenylene oxide, and polyurethane) or elastomeric polymers (e.g. various Silicones, natural rubber, butadiene-styrene rubber, carboxylic butadiene-styrene, butadiene-acrylonitrile rubber, carboxylic butadiene-acrylonitrile rubber, chlorobutadiene rubber, polybutadiene rubber, silicone rubbers, and acrylate rubbers, perhaps vulcanized, and other elastomeric materials) or a composite material.

The material used to join the segments may also have additional biological or mechanical properties. For example, the material may incorporate a therapeutic or medicinal agent for release (e.g., timed release). Examples of therapeutic agents include, but are not limited to, antibiotics, analgesics, anticoagulants, bone growth enhancing agents, cells or gene therapies, etc. The material may also incorporate other agents and materials, for example, radiopaque materials to aid visualizing the implant.

The joining material may also be severable. It may be desirable to have implants of certain lengths (e.g. a certain number of segments). It may also be desirable to have implants that are continuous, and allow the user to select their length by removing or cutting the connection between any two segments. For example, the joining material may be severable by mechanical, thermal, chemical, or electrical means.

In one variation, the joining material is removable from some or all of the segments during or after insertion into the cavity.

Joining Material as Flexible Tube

In the variation of the implant shown in FIG. 1E, the segments are linked together in linear array because they are held within a flexible tube 19. A flexible tube may be made of virtually any material, so long as the final implant is adequately flexible to allow bending of the implant. The flexible tube comprises a solid or continuous walled tube, a solid or continuous walled tube having openings in the wall, or a netting woven from string or fiber. The flexible tube may comprise one or more membrane, optionally made of an expandable or a stretchable material.

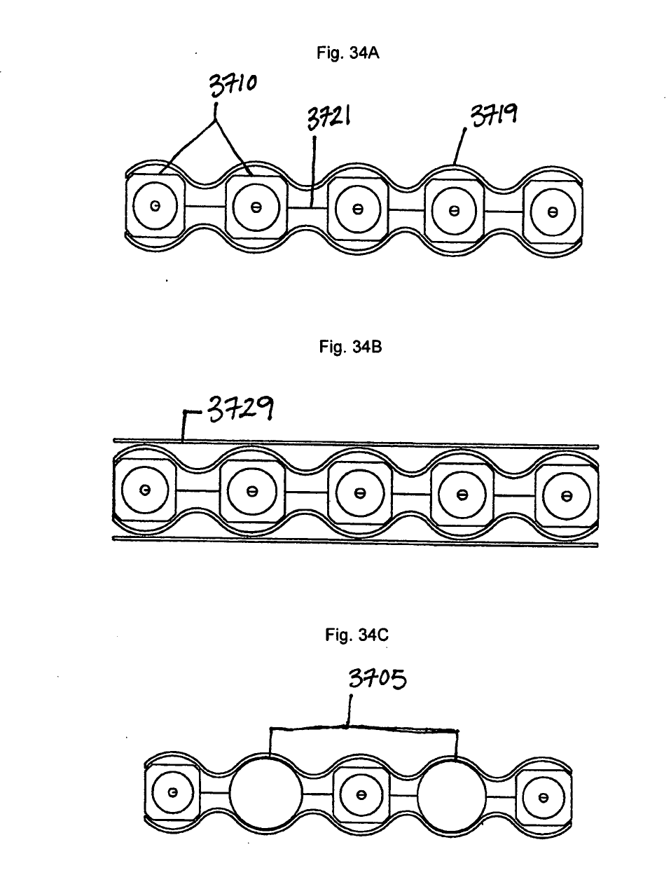

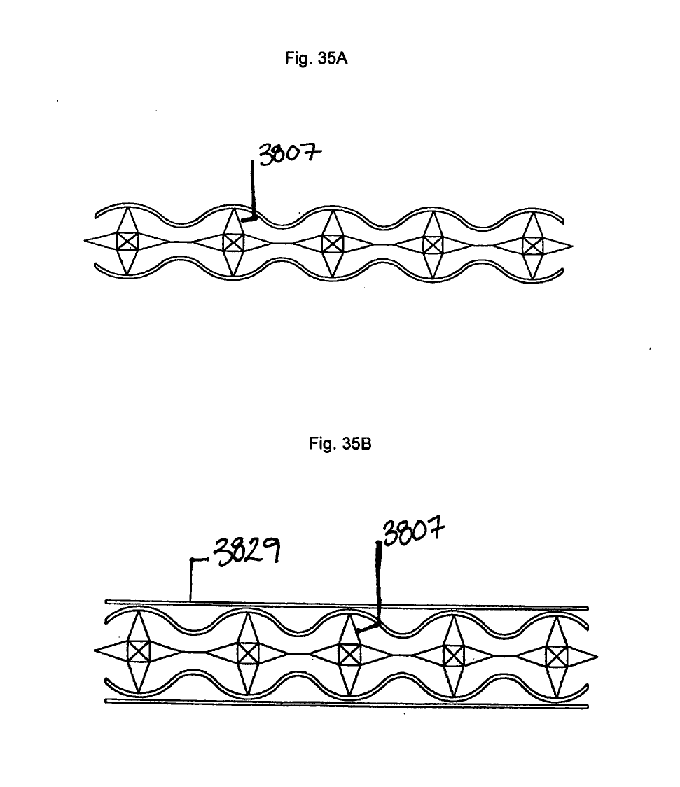

In one variation, the implant segments are linked by an expandable membrane. The expandable membrane material may be a fabric that has pores allowing passage of fluids and bone growth through it. For example, the membrane could be formed of a flexible polymeric fabric e.g., high molecular weight polyethylene. The flexible tube may be any material (e.g. woven, non-woven, extruded, etc) that is adequately flexible. In FIG. 34A, one variation of the implant has segments 3710 that are within the flexible tube 3719 that are also linked by a filament, wire, string, or other connecting or joining material 3721.

In one variation shown in FIG. 34B, the flexible tube with segments is located within a second flexible tube 3729. The internal flexible tube may contain perforations to allow the passage of fluids into the outer flexible tube. The internal flexible tube may contain variations that allow the passage of fluids into the outer flexible tube only after the implant is delivered into the non-soft tissue cavity. For example, an activating agent may make one or both of the flexible tubes porous (e.g., by adding a solvent such as water or other fluent material). In one variation, the passage of fluids between the flexible tubes (or from the flexible tube into adjacent body regions) occurs after compaction or after a specific geometry (e.g. bond angles) or position of the contained chain of segments is achieved. The inner flexible tube may be enclosed by any number (e.g., two or more) flexible tubes.

In one variation shown in FIG. 34C, fluent materials such as cements are contained within one or more segments within the flexible tube 3705. These specialized segments might be a crushable material that allows the release of the fluent material (such as a cement) into the flexible tube that can then interact with a secondary hardening catalyst located within the tube and react to begin setting to form a hardened final composite. This process would allow the settable material to harden within the tube after desired placement and packing of the flexible tube implant. In one variation, the flexible tube membrane might be permeable allowing some of the hardening fluent settling material to move to the external space surrounding the flexible tube. In one variation, the flexible tube would be impervious to the fluent material (e.g., no porous) and the cement would remain fully contained within the flexible tube.

In one variation shown in FIG. 35A, the segments within the flexible tube may include sharp protrusions 3807 that allow the perforation of the flexible tube during or after delivery of the implant. This would allow the two fluent settling materials to mix and begin the hardening process after or during delivery of the implant In the variation shown in FIG. 34B, the flexible tube includes some segments with sharp protrusions and is contained within a second outer flexible tube 3829 that could contain a settling material released from the internal flexible tube. The sharp edges or protrusions may be caused to pierce the inner tube (and possibly the outer tube) in a controlled manner (e.g., by compaction).

In one variation, the flexible tube might be coated with a bonding agent. The bonding agent may allow adhesion of the implant to bone or other non-soft tissue within the cavity. The bonding agent may allow adhesion of the implanted flexible tube to itself. The bonding agent may allow adhesion to both. The bonding agent might be coated onto flexibly connected segments that are not contained within a flexible tube.

In one variation, two or more flexible tubes may be delivered simultaneously from within one delivery cannula. In one variation two or more chains of flexibly connected segments may be delivered simultaneously within one flexible tube. In one variation, two or more chains of flexibly connected segments, each individually enclosed within a flexible tube, may be contained within one outer flexible tube and may be delivered simultaneously within a delivery cannula. The segments contained within the flexible tube or tubes may be non-connected except by the flexible tube. In one variation two or more chains of segments may delivered without a flexible tube within one delivery cannula.

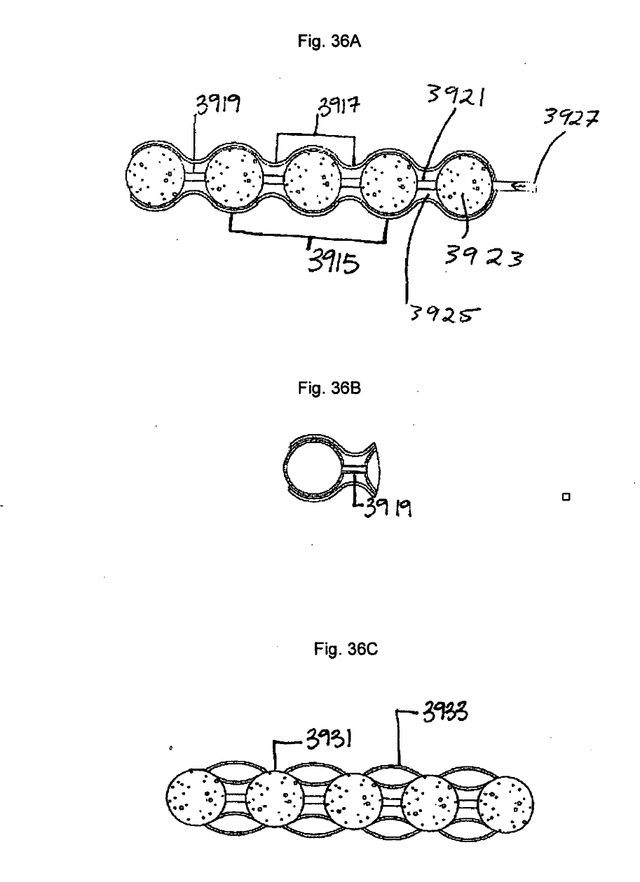

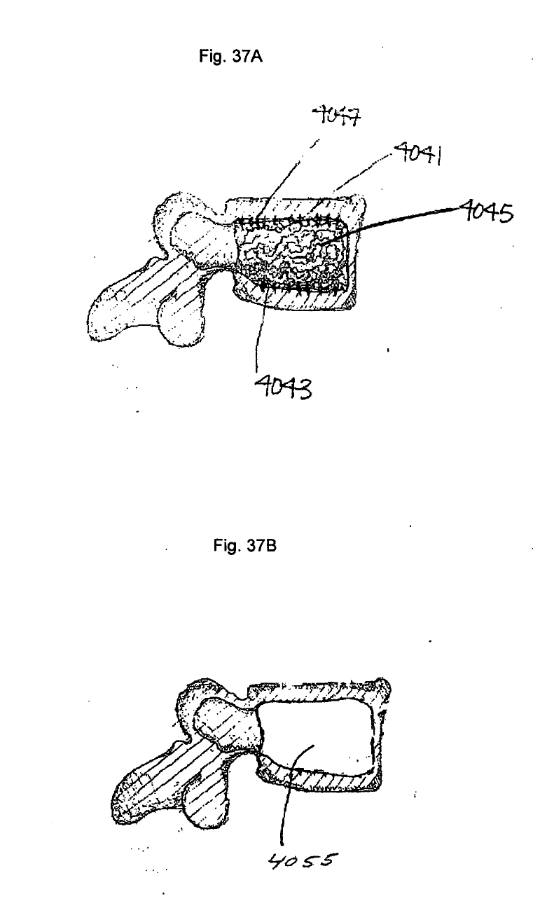

The implants described herein may also include one or more transmission pathways for transmitting electromagnetic energy (e.g., light such as UV light, electrical or magnetic energy, etc.). This electromagnetic energy may be used to activate a fluent material within the implant (or adjacent to the implant), causing it to harden. For example, in one variation (as shown in FIG. 36A), the flexible tube contains electromagnetically transparent segments 3923 and connectors comprising a material that transfers electromagnetic energy (such as a fiber optic material 3921). Other types of electromagnetic energy that might be utilized could include gamma rays, infrared, x-rays or ultraviolet waves. In one example, the segments and connecting material can be surrounded by a fluent, UV-curing, settable material 3925, such as an epoxy, resins, polymer, monomer, or an acrylic, that is capable of being hardened upon exposure to an electromagnetic energy transferred through the connecting material, such as a fiber optic 3927. Examples of UV-curing materials include UV curable adhesive& potting compounds such as UV Cure 60-7155 (a one-component modified epoxy) and DYMAX UV resins. The use of UV-curing materials can control when the hardening of the settable material begins, providing additional control of delivery of the implant. The transparent segments might be composed of a polymer or any material that is capable of transferring light. The UV-curing material might be utilized in any cavity space where a controlled timing of delivery of a settable material is desired. For example the cavity might be a intra-vertebral space such as is shown in FIG. 37B. In one variation, the electromagnetic energy can be transferred through the sheath itself, which could be composed of a material capable of conducting the electromagnetic energy. Any appropriate transmission pathway may be used, including dedicated pathways (e.g., fiber optics, conductive wires, etc.) or pathways made of the segments and/or connecting filaments, the tube, or even the fluent material itself. In one variation, the segments within the flexible tube are coated with a material that can have a phase change when catalyzed by an electromagnetic energy. In one variation, a fluent or coating material may be catalyzed to harden (phase change) or become adhesive by the application of heat from a heat source (e.g., laser, electrical resistance, etc.). In one variation, the transfer of energy within the implant might be guided by making transfer from the implant, or in certain regions of the implant, inefficient. For example, some of the surfaces of the implant (e.g., within the tube or the segments) may include a surface finish treatment or coating to reflect or inhibit electromagnetic energy, distributing the energy within the implant in a predictable way.

In one variation, an inner flexible tube contains segments and a fluent settable material (such as cement) and an outer flexible tube surrounds the inner flexible tube. The space surrounding the inner flexible tube contained within the outer flexible tube might contain a biologic bone growth material such as bone morphogenic protein. In one variation, the inner flexible tube is porous. In one variation the inner flexible tube is not porous (e.g., impervious to the passage of fluent material such as activatable cement). In the variation shown in FIG. 36A, the flexible tube contains porous regions. The flexible tube might be porous at the point of contact with the segments contained within the tube 3915 but not-porous along the rest of the tube 3917. As shown by FIG. 36B the segments might be connected within the flexible tube by small tubing 3919 that allows the passage of fluent materials between the segments. In one variation the flexible tube might be completely porous. In one variation shown in 36C the connecting member may be composed of one or more flexible tubes between the segments 3933 allowing a portion of the segment to be exposed directly to the cavity space such as a vertebral space 3931.

Multiple implants (e.g., including implants with different properties) may be used in the same procedure. In one variation shown in FIG. 37 a single chain of flexibly connected segments is placed along the top of a non-soft tissue cavity 4041 such as a vertebral space and one chain of flexibly connected segments across the bottom of the tissue cavity 4043. An implant of flexibly connected segments contained within a flexible tube may then be delivered into the cavity between the two individual chains 4045. In one variation, the individual segment chains are shaped in a manner that would cause them to penetrate the non-soft tissue 4047 such as bone endplates within a vertebra space resulting in the chains becoming secured.

In one variation, the flexible tube contains segments and is designed to create a void within a non-soft tissue cavity. In one variation, the non-soft tissue is bone tissue. In one variation, the tissue is cancellous bone tissue. The flexible tube may be removed after the void is created.

In one variation, a flexible tube containing segments is delivered into a cavity such as a vertebral space to create a void within the space, and is then removed. A small amount of fluent adhesive material (such as cement) is applied to internal non-soft tissue such as the top and bottom bony endplates within a vertebra. A subsequent implant of flexibly connected segments contained within one or more flexible tubes is then delivered into the cavity. This implant may include a fluent settable material such as cement along with the segments within the flexible tube at the time of delivery into a cavity such as a vertebral space. In one variation, the secondary flexible tube implant is delivered without a void being created in advance.

Segment Dimension

FIGS. 1A to 4D show different variations of the segments 12 compatible with the implant 10. In FIG. 1 the segments are all shown as spherical pellets. FIG. 1B shows that the pellet size may vary. FIG. 1C shows that the spacing of the segments on the joining material (shown as a filament 16) may vary. The lengths of the implant (e.g. number of pellets) may also vary. Larger 14 segments and smaller 18 segments are arranged in the linear array. Virtually any combination of segment sizes and shapes may be used in the implant. Varying the size as shown in FIG. 1B may change the manner that the implant “packs” within a bone cavity. For example, packing of different sized segments may allow different spacing between the segments, and therefore different opportunities for tissue in-growth into the implant, different structural properties, and different loading patterns of adjacent structures.

Segmented implants may be configured so that the implant is securely packed into the body region (e.g. non-soft tissue cavity). Size, shape, and spacing all contribute to the packability of the implant within the body region. For example, the same implant may have segments of different sizes, shapes and spacing in order to optimize packing. Additional factors such as the ability of one or more segments to move along the linear axis of the implant may also contribute to packing.

The size of the segments may be selected to optimize the insertion into the cavity and use of the implant applicator described below. Thus, the segments may describe a range of sizes suitable for use with an applicator and/or suitable for insertion into a bone cavity of given dimensions. In one variation the segments are between 1 to 40 mm in diameter. In one variation the segments are between 1 to 37 mm in diameter. In one variation the segments are between 1 and 10 mm in diameter. In one variation, the segments are between 1 and 6 mm in diameter. In one variation the segments are approximately 3 mm in diameter. In one variation the segment diameter is an average segment diameter. In one variation, the segment diameter is the maximum diameter of a segment.

The implant may have different inter-segment spacing. FIG. 1C shows implant segments 12 arranged in a linear array in which there are larger 20 gaps and smaller 22 gaps between adjacent segments. Different arrangements of segments along the linear array may also have desirable effects on the packing behavior of the implant and the severability of the implant. FIG. 1D shows a variation of the implant in which the spacing between segments is extremely small 24, potentially reducing the flexibility of the implant. However, implant flexibility may also be increased by using more elastic joining materials and potentially allow greater packing.

The segments may also be slideable (or partially slideable) in one (e.g. the long or linear) axis of the implant. In one variation of the implant some of the segments are slideable and some of the segments are fixed to the joining material. In at least one variation of the implant, the slideable segments allow the implant to be “tensioned” by tightening the joining material, tending to stiffen the implant, perhaps to aid in anchoring the implant or distracting a bone separation, or in anchoring another implant or device.

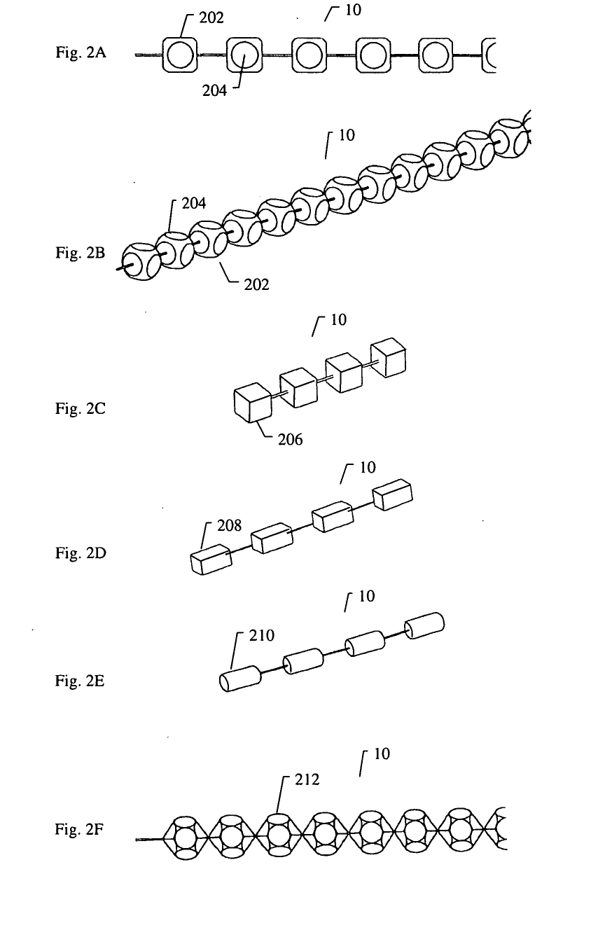

The segments of the implant may also have different shapes, allowing different packing and implantation properties. FIG. 2 shows examples of segments with different shapes. FIGS. 2A and 2B show a schematic and perspective view of cubic segment 202 shapes with rounded edges. The parallel faces of these segments 204 allow closer packing between adjacent segments. FIG. 2C is also an implant with cubic segments 206. FIG. 2D shows an implant with rectangular-shaped segments 208. FIG. 2E shows an implant with cylindrical segments 210. FIG. 2F shows an implant with a slightly more complex segment shape having more than six faces. Virtually any shape that will allow the implant to fill a cavity to distract a cavity, create a cavity, and/or tighten or secure another implant, may be used. As used herein, unless the context makes it clear otherwise, “fill” means that the bone cavity is supported in three dimensions.

The implant assemblage described herein describes space-filling implants (for filling, distracting, void creation, etc.). Thus, implant segments may be adapted specifically to fill three dimensional spaces.

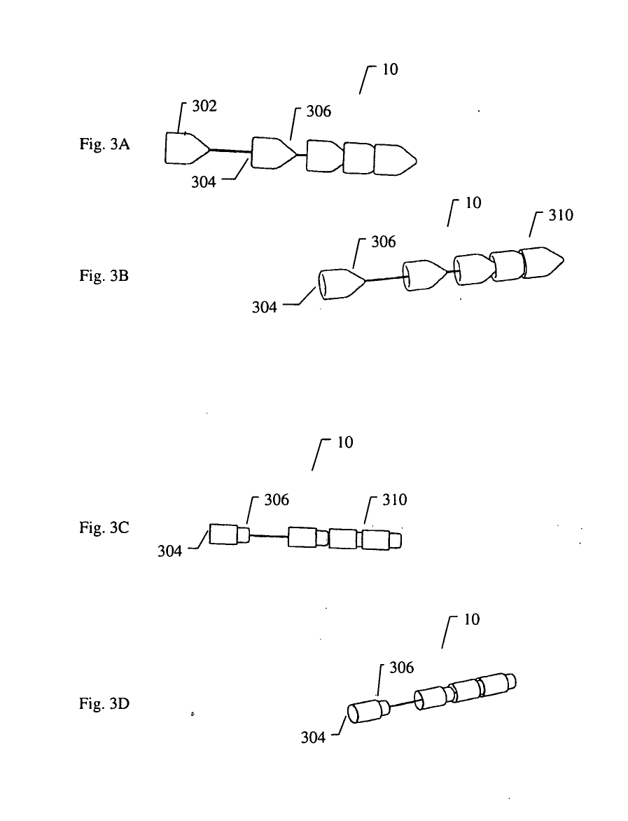

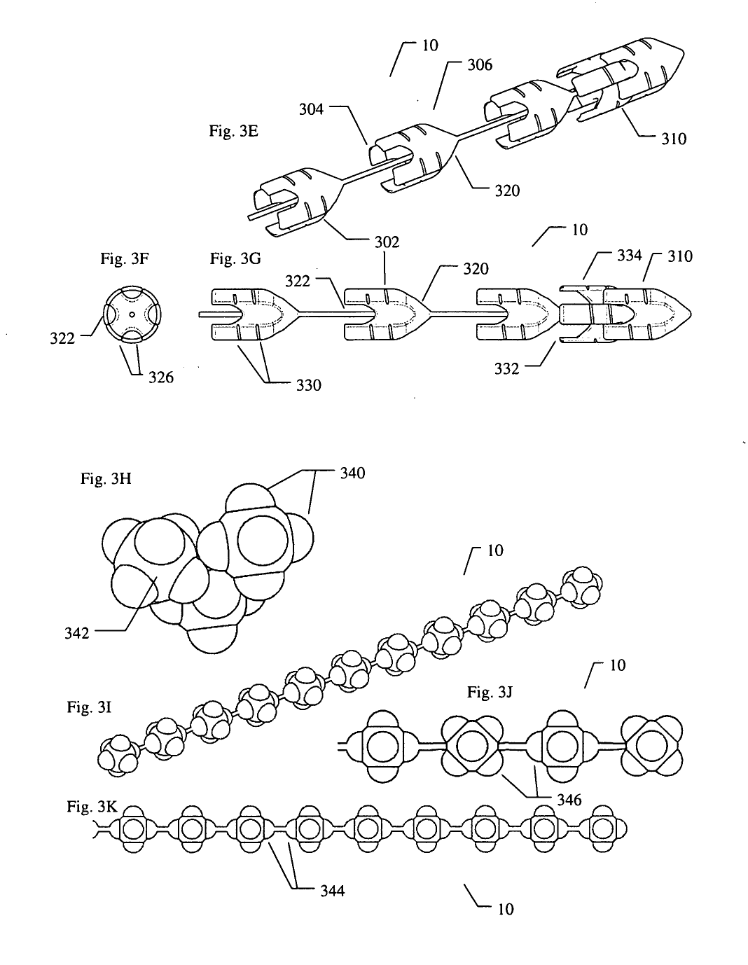

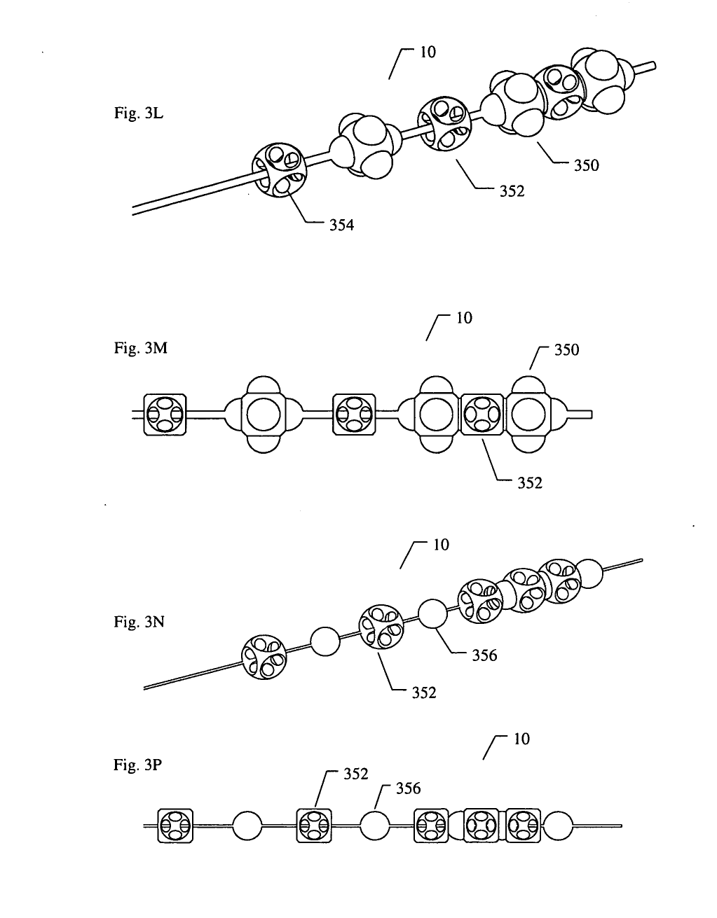

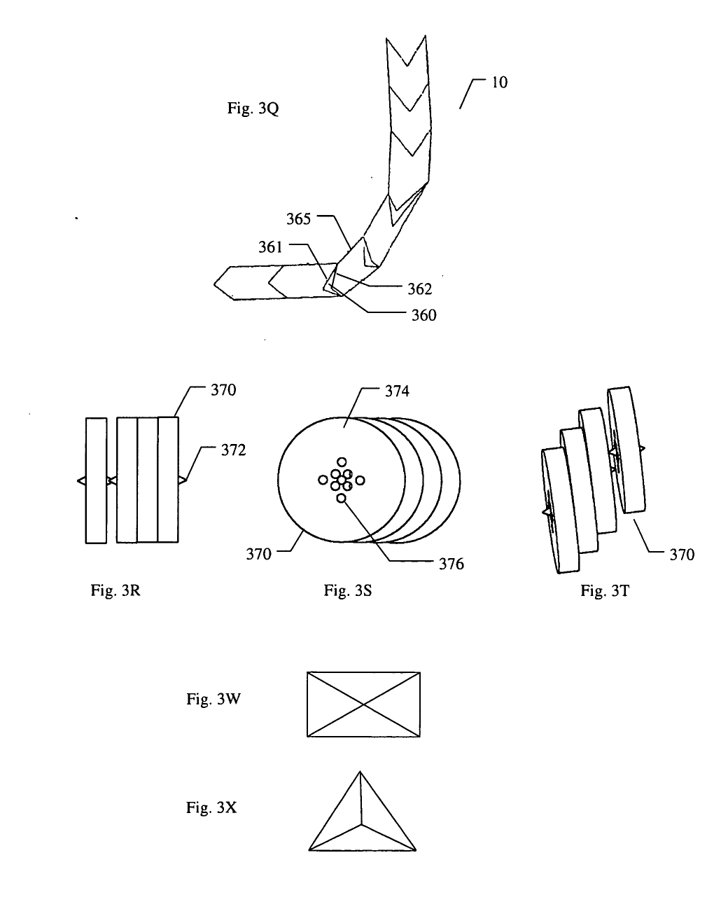

The implant may have segments of different shapes, including shapes that are configured to communicate with each other, for example, to interlock. Several examples of interlocking shapes are shown in FIG. 3A to 3X. In FIG. 3A to 3G, the bullet-shaped 302 segments have a front end 306 and a back end 304, and at least some of them may slide along the axis of the linear array of the implant 10. The back end of one segment can engage with the front end of an adjacent segment as shown 310.

The segments may also be shaped to engage non-adjacent segments, for example, by having side faces that engage with other segments. The segments may also be shaped to engage with the walls of the cavity.

In FIG. 3E to 3G, the segments have a bullet shape with a conical nose 320, a cylindrical body 322, and a conical recessed rear 324, with linear and rotational inner-locking features, 326. FIG. 3F shows a frontal view of two segments interlocked; FIGS. 3E and 3G show linked segments. The external surface has an advancing helical ramp 330 for assistance in advancement of a segment relative to adjacent segments when an axial load and rotational load are simultaneously applied to the implant. These features aid in compacting and elevating the hard tissue around the cavity being filled. The flexible rear extension 334 with external round 332 increase the likelihood of interstitial placement.

In FIGS. 3H to 3K, the implant comprises common segment shapes that have six over-lapping male spherical ball geometries creating a complex external multiply spherical surface 340. FIG. 3H shows three segments interacting. FIGS. 3I to 3K show linked segments. These segments may interlock because of the spheres nesting within the adjacent segments' depression created by the curved (e.g., semi-spherical) segment surfaces creating multiple coincident mating tangency points 342. The segments can be arranged along the connective member in a common entry and exit orientation 344 as in FIGS. 3I and 3K or an alternating pattern 346 as in FIG. 3J.

In FIGS. 3L and 3M, the implant 10 consists of two different segment shapes alternating and repeating along the connective member. The first segment 350 is similar to the segment described in FIGS. 3H to 3K consisting of six over-lapping male spherical ball geometries 340. The second segment 352 is a segment that has six female spherical recesses 354 that will enable tight interlocking and packing of the implant within the cavity.

In FIGS. 3N and 3P the implant 10 consists of two different segment shapes alternating and repeating along the connective member. The first segment 352 is similar to the segment in FIGS. 3L and 3M. The second segment 356 is spherical. The configuration of this implant affords a tight packing with numerous mating receptacles open to accept the spherical segments and thus may be less dependent on packing order than other variations.

In FIG. 3Q, the implant 10 consists of two different segment shapes alternating and repeating along the connective member. The first segment 360 is arrowhead-shaped with front 361 and rear faces 362 pointed and made up of two angled faces. The second segment 365 is an elongated arrowhead with otherwise similar front and rear faces. The segments can be arranged in a manner that will allow a control of the desired mating and direction that the segments will follow once the segments leave the delivery cannula and meet resistance within the cavity. The direction change will be dictated by slight angular differences between the mating arrowheads.

In FIG. 3R the implant comprises common segments shaped like coins 370 with conical spikes 372 protruding from the faces of the coins. The coin faces 374 have holes through them 376 that facilitates stacking of the coins, and the spikes are conically shaped to facilitate the self-centering stacking of the segments. The stacked coins create common tangency points 180 degrees opposed from each other that create two parallel planes of support.

In FIG. 3W the segments have a cross-sectional area that is rectangular with various previously described front and rear geometries.

In FIG. 3X the segment cross-section is triangular with various previously described front and rear geometries. In some variations, the segments can have polygonal cross-sections, for example, hexagonal, octagonal, etc.

The aspect ratio of the segments' length relative to the segments' height and width can be varied in order to allow variations of stacking, packing, steering or elevating, depending on the desired result.

Many of the implant segments shown (e.g. FIGS. 1, 2 and 3A-3K and 3Q-3T) are illustrated as substantially ‘solid.’ Implant segments may also be hollow or have passages for either the joining material or additional material such as a fluent material (e.g. cement). Implant segments may also be porous, for example, to facilitate tissue in-growth, or reduce overall segment weight. FIGS. 4A and 4B show an implant that has passages 402. FIGS. 4C and 4D show an implant with pores, or hollow spaces, 404 that do not span the length of the segment. In one variation the pores 404 are dimples.

Implant segments may also be used with a fluent material. Examples of fluent materials include cements (e.g. bone cements, synthetic bone graft cements, etc.), therapeutics (e.g. bone morphogenic proteins, cells or gene therapies, bone growth factors), or combinations or substitutions thereof. In one variation the fluent material is applied into the cavity after the implant has been inserted. In one variation the fluent material is added before the implant. In one variation, the fluent material is added concurrent with insertion of the implant. In one variation the fluent material is inserted into the flexible joining material (e.g. a flexible tube around the implant segments). The flexible tube may be impermeable to the fluent material, keeping it substantially contained within the bone cavity.

Applicator

An applicator may be provided to insert a material such as the implant into a cavity to fill or distract the cavity, and/or to create or expand a cavity. The applicators described herein may be used to insert or remove an implant described herein. The applicators described herein may be used with any compatible material, including but not limited to individual pellets, fluent materials, and linear arrays of any materials desirable for insertion or removal from the body.

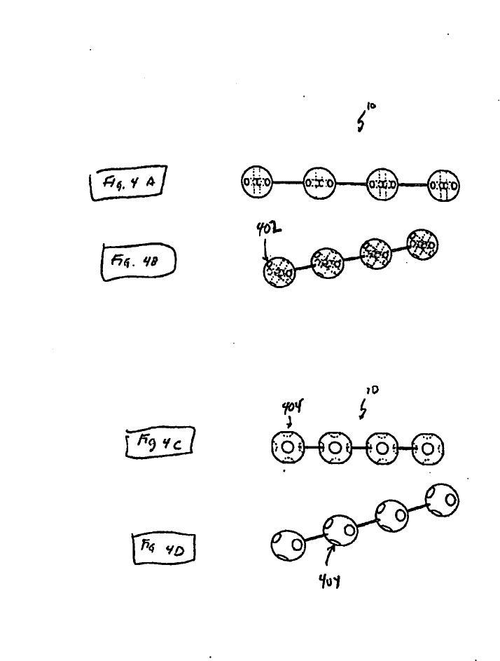

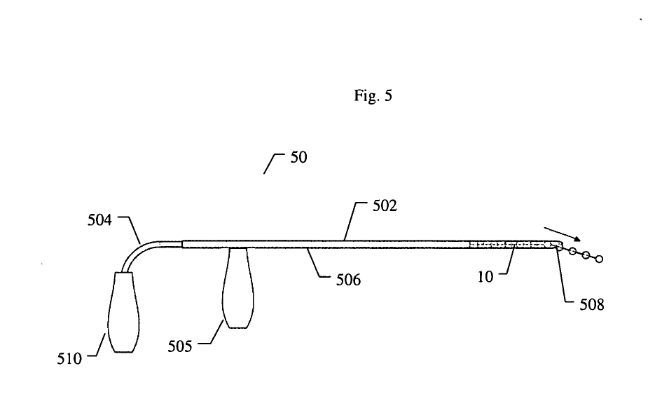

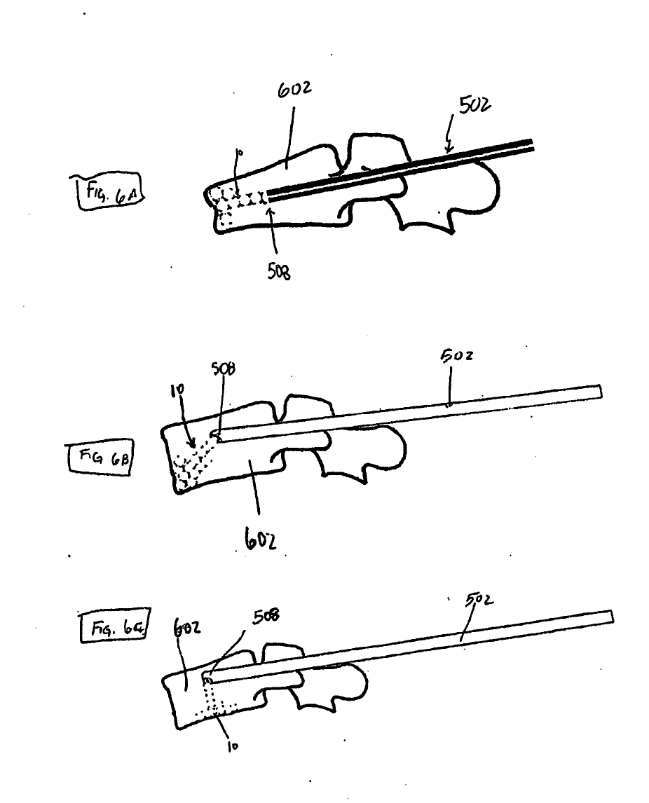

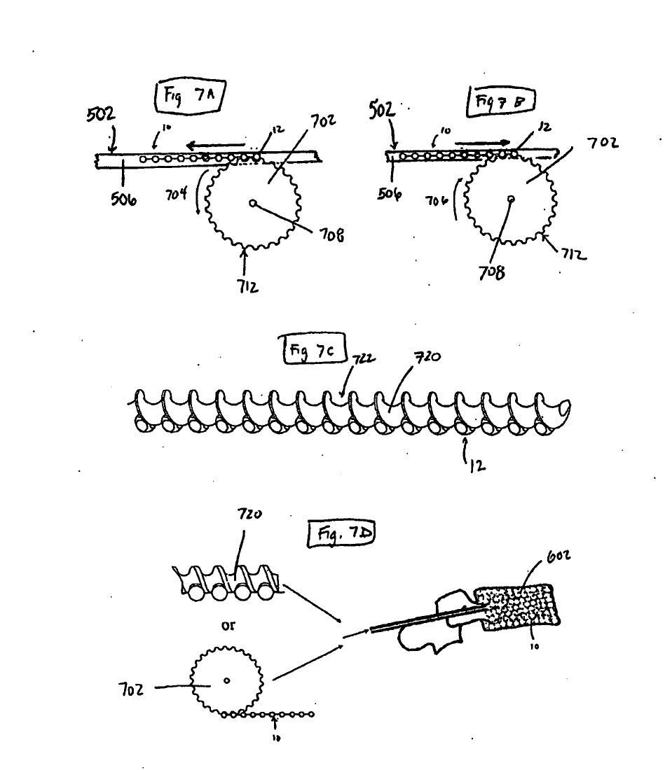

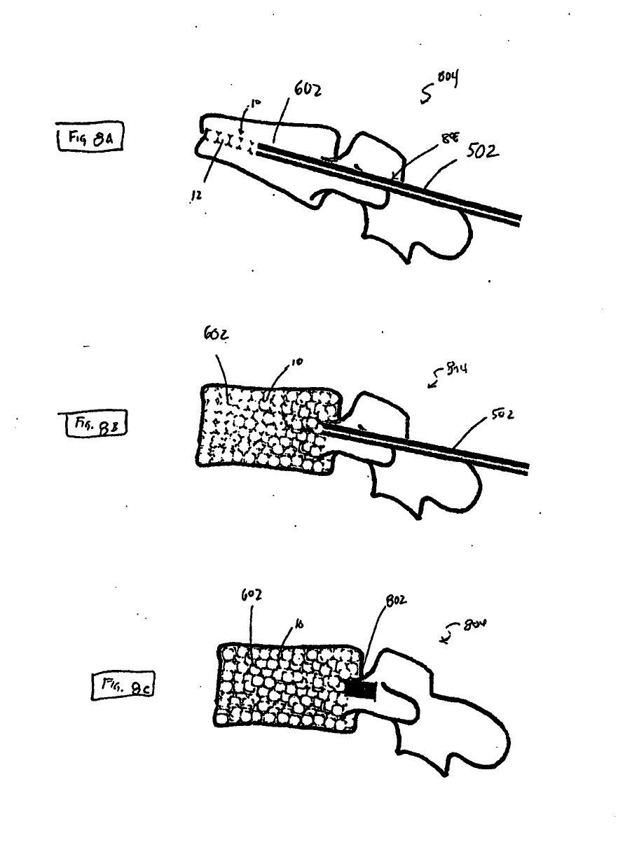

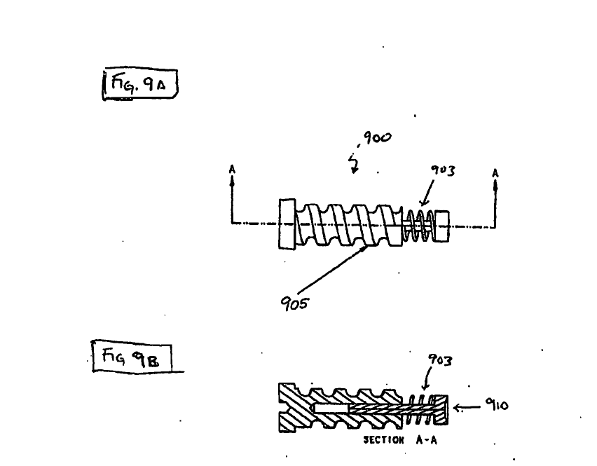

FIG. 5 shows an applicator 50 useful for inserting an implant into a cavity (e.g. a bone cavity). The applicator has a cannula 502 having a distal and a proximal end and a lumen 506 with a handle 505 to aid in controlling the distal end orientation of the cannula. An implant 10 can be inserted into a bone cavity from the distal end of the cannula through an opening at the distal end 508. A feed guide 504 connects to the proximal end of the cannula. The feed guide can insert or withdraw the implant in and out of the lumen of the cannula through an opening in the proximal end of the cannula. An applicator may also have a handle 510 or a feed chamber to store implant material.

Cannula

The cannula may be an elongated tubular member having a lumen or passage to facilitate the movement of an implant through the cannula. The inner lumen of the cannula may be configured to hold and allow the passage of an implant. The inner surface of the lumen may be size-matched to the diameter of the implant. Alternatively, the size of the implant (e.g. segment size) may be limited by the inner diameter of the applicator cannula. The inner surface of the cannula may include a material that facilitates the movement of an implant (for example, a friction-reducing coating or a lubricant). The cannula may also allow the passage of a secondary filling material (e.g. a fluent material) before, after and/or during the insertion of an implant. An applicator cannula may be flexible or rigid.

The cannula may also have a fastener towards the distal end to hold the cannula in place on the outer surface of the bone being treated. A fastener or gripper near the distal end of the cannula may be used to aid the user in holding an applicator steady while inserting the implant to distract a bone cavity. In one variation the distal end of the cannula is threaded to facilitate insertion into, for example, the pedicle of a vertebra. The threads may further serve as a fastener or gripper.

The distal end of an applicator cannula may be adapted to aid in penetrating and/or distracting a bone cavity. In one variation, the distal end of the cannula includes a trocar. In one variation, the distal end of the cannula includes a spreader to separate bone surfaces and aid insertion of an implant.

The distal opening of an applicator cannula may be located at the distal-most part of the cannula, or it may be located all or partly on the perpendicular axis of the cannula (e.g. on the side of the cannula, or at an angle), allowing more directional filling of a bone cavity by an applicator. FIG. 6A shows the distal end of an applicator cannula in which the distal opening is the extreme distal end of the cannula. The implant 10 exits the applicator 502 through the cannula's distal opening 508, and begins to fill the bone cavity 602, as shown.

FIG. 6B shows the distal end of an applicator cannula in which the distal opening 508 is at a 45° angle from the long axis of the cannula. Thus the implant 10 is inserted into the bone cavity 602 at a 45° angle relative to the cannula. FIG. 6C shows the distal end of an applicator cannula in which the distal opening 508 is at a 90° angle from the long axis of the cannula. Thus the implant 10 is inserted into the bone cavity 602 perpendicular to the cannula.

The outer surface of the cannula may have graduated indicia that provide depth of penetration information during insertion by the user.

An applicator may be operated with a guide cannula. In one variation, an applicator cannula fits into the lumen of a guide cannula; the guide cannula is used to locate and prepare the bone cavity for insertion of the implant by an applicator. In one variation, an applicator cannula locks into a guide cannula and the guide cannula is secured to the bone that is being operated upon.

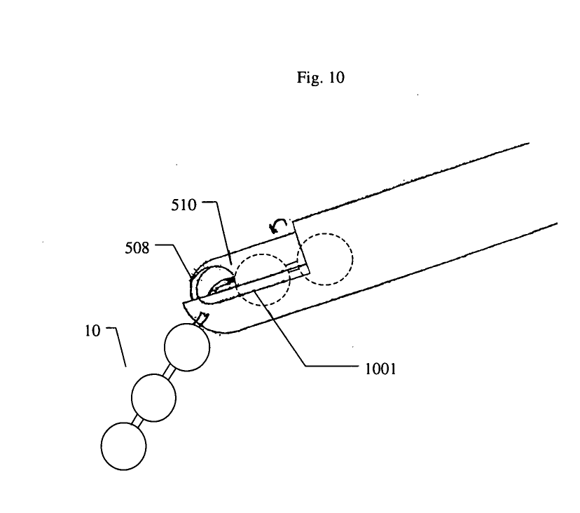

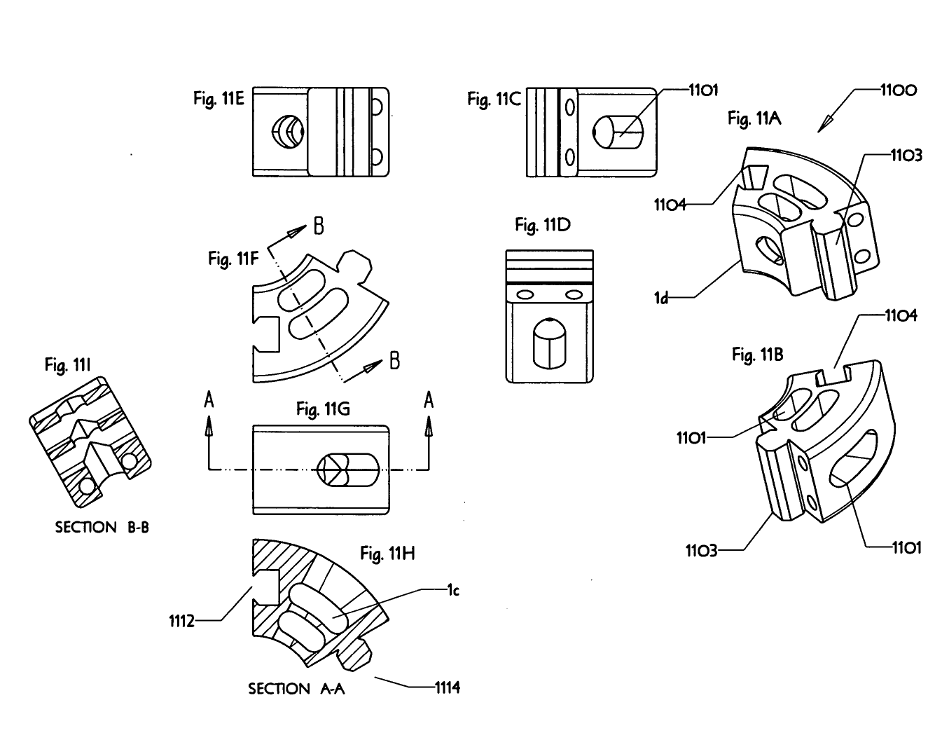

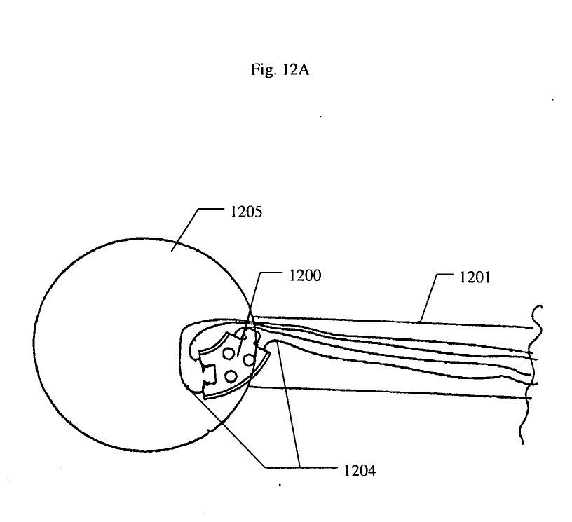

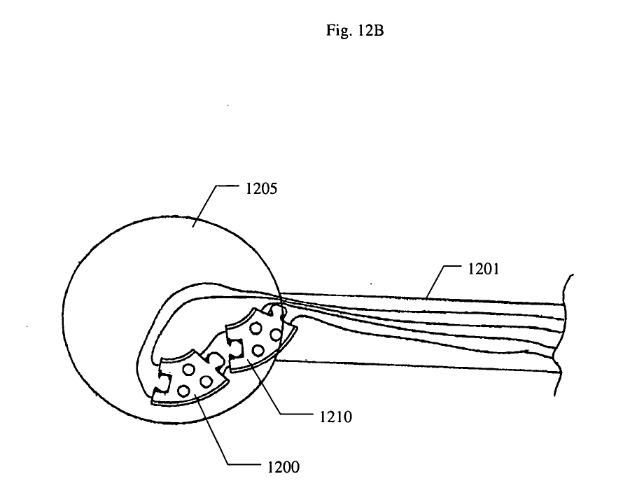

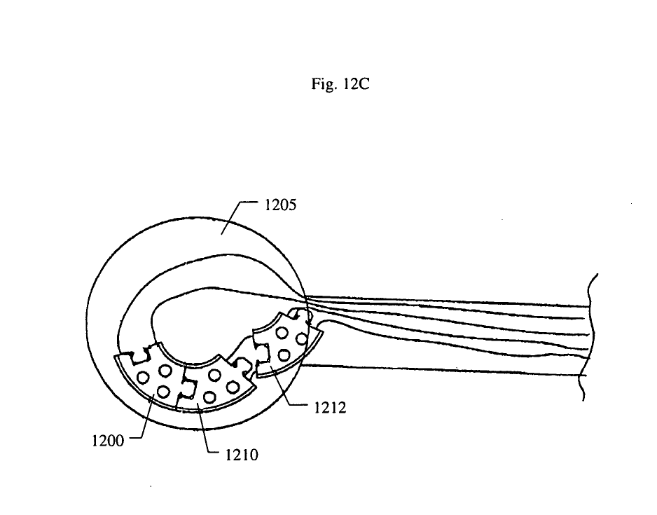

An applicator may also include a cutter configured to sever the implant by removing the connection between two of the segments in the linear array of an implant. An example of a cutter 1001 is shown in FIG. 10. The cutter may be located at least partly at the distal end of the cannula. The cutter may be located at least partly within a region of the inner lumen of the cannula. In one variation the cutter is located at an outer surface 509 of the distal end of an applicator cannula, adjacent to the distal opening 508. Rotating an external sheath drives a cutting edge across the cannula's distal opening thereby severing the connection between implant segments. In this variation the cutter is actuated by rotating the external sheath 510. As illustrated in FIG. 10, the cutter may be a mechanical cutter capable of applying force to sever the implant. Additional examples of mechanical cutters include but are not limited to, a blade, a scissor-like cutter, and the like. The cutter may be an electrical cutter capable of applying electrical energy to sever the implant. The cutter may be a chemical cutter capable of chemically severing the implant, for example, by applying a compound that reacts with the joining material of the implant. The cutter may be a thermal cutter which acts, for example, by heating the material connecting the segments causing it to release. The cutter may be any combination of mechanical, electrical, chemical and thermal cutter. The cutter may be controlled by a cutting controller. The cutting controller may be controlled directly by the user, or as part of a system.

Driver

An applicator may further comprise a driver for applying force to the implant in order to move the implant within the cannula to insert the implant into or withdraw the implant from a bone cavity. An applicator may be a mechanical drive (e.g. linear driver, a rotary driver, etc.), a pneumatic driver, hydraulic driver, a magnetic driver, an electric driver, or any combination thereof. Examples of drivers include, but are not limited to, rotating auger drivers, and rotating cog drivers. The driver is preferably a rotatable driver. Force generated by the driver is transferred to the implant (or a part of the implant), moving the implant within the cannula, in either the proximal or distal direction. In one variation, the driver is located at least partly within the cannula. In one variation the driver is located at least partly within the feed guide. An introducer member may comprise a driver as described here.

Applicator drivers engage at least a region of an implant. FIGS. 7A and 7B illustrate a cog driver 702 engaging at least part of an implant 10. As the cog is rotated about its central axis 708, in the direction indicated by the arrows (704 and 706), the implant is moved in the complimentary direction because segments of the implant 12 have engaged with the cog teeth 712 and are pulled or pushed in the direction of the rotation as shown. Because the segments of the implant are connected, movement of at least one of the segments results in moving the implant. An applicator driver may comprise more than one cog, or a cog and other driver components. FIGS. 7A and 7B also show the driver (a cog) at least partly in the lumen 506 of the applicator cannula 502.

In one variation, the cog is a friction wheel. In one variation, an outer surface of the friction wheel driver engages one or more regions of an implant (e.g. a segment). When the cog is a friction wheel, it may not have “teeth” which engage the implant.

FIG. 7C shows a rotating auger driver. In one variation, the auger is a continuously threaded rod 720; the implant's segments 12 fit within the threading gaps 722. In one variation, the rotating auger is located at least partly within the cannula. At least some of the implant segments are seated in the auger and are prevented from rotating around the long axis of the auger, for example by the geometry of the cannula or chamber surrounding the auger. Rotating the auger forces the segments (and thus the implant) to move down the long axis of the rod. Reversing the direction of rotation of the auger changes the direction that the implant moves. An applicator driver may comprise more than one auger, or an auger and other driver components.

A driver may also be at least partially within the cannula. In one embodiment the cannula lumen contains a rotatable auger. In one variation the driver is entirely located within the cannula.

A driver may be located at the proximal end of the applicator cannula, as indicated in FIG. 7D. Force applied by the driver moves an implant within the cannula, into or out of the bone cavity 602. The driver may be capable of moving an implant into or out of a bone cavity by changing the direction that force is applied to the implant. An applicator driver may be attached to, integral to, or coupled to a feed guide.

Feed Guide

An applicator may include a feed guide 504 for loading the applicator cannula with an implant. A feed guide may be coupled to the proximal end of the cannula as shown in FIG. 5. A feed guide may comprise a chamber, a cartridge, a track, or other such structure in which an implant can be held. The feed guide may orient the implant for inserting or withdrawing from the cannula. The feed guide may also assist in engaging an implant with a driver.

In one variation, a feed guide is preloaded with an implant. For example, it may be advantageous to have the feed guide be a pre-loaded cartridge holding an implant. Such a feed guide may be separately sterilized and interchangeable between applicators.

In one variation, the feed guide includes a track configured to guide an implant. A track may keep the implant from jamming or tangling within the applicator. A track may further allow a long implant to be stored compactly. The feed guide may also help regulate the amount of force needed to move the implant.

In one variation the feed guide may be configured to engage an implant into a driver. In one variation a driver is at least partly contained within the feed guide. In one variation the feed guide attaches to a driver. In one variation the feed guide is configured as an opening in the cannula into which an implant may be manually inserted.

Controller

An applicator for inserting an implant may also include a controller for controlling the applicator driver. A controller may be manually or automatically operated. A controller may control the force applied by the driver. The controller may control the rate of insertion/withdrawal of an implant. A controller may control the direction that force is applied (e.g. forward/reverse). A controller may be operated by a user.

An applicator may also include detectors or indicators for registering implant and applicator parameters. In one variation an applicator includes a detector for determining and/or indicating the force applied by the applicator to insert or withdraw an implant. When a cavity is being filled, and particularly when a bone cavity is being distracted, an implant may be applied using a force adequate to insure that the implant is properly positioned within the cavity. Thus it may be important to monitor force and pressure applied to the implant or volume of implants, and/or the tissue. Feedback mechanisms may also be used to regulate the actions of the applicator, including the force applied by the applicator.

An applicator may also include detectors or indicators for indicating the status of the implant. For example, a sensor may indicate the amount of implant inserted, the amount of implant left in the applicator, and/or the position of the implant within the applicator or the bone cavity. In one variation, the applicator includes a force gauge for detecting the force applied by the applicator on the implant being inserted. The applicator may also include a display capable of indicating a status. Examples of the kinds of status that the display could indicate include, but are not limited to, force applied, total volume, linear feed rate, volume feed rate, amount of implant material inserted, and/or amount of implant material remaining in the applicator.

Implants Compatible with the Applicator

The application described herein may be used with any compatible implant, including but not limited to discrete (loose) pellets or segments of any material (including segments or pellets as described herein), fluent materials (e.g. cements, bone fillers, etc.), and any implant, particularly those comprising a linear array of elements. Such applicators may also be useful for filling and distracting bone cavities. In one variation the applicator comprises a cannula and a driver where the driver further comprises an auger or a cog. The auger or cog propels the discrete pellet, fluent material, or combination of implants, discrete pellets and/or fluent material, down the cannula in order to fill or distract the cavity into which the cannula has been inserted. It may be particularly advantageous to use the applicator with flexibly connected implants, including those described herein, because the applicator may be used to controllably insert and remove flexibly connected implants.

Additional exemplary applications of the applicator and/or implants as described herein are given below. These examples are intended only to illustrate various embodiments of the implant, applicator, and methods of use, and are not intended to be in any way limiting.

### EXAMPLES

In general, the implants and/or applicators described herein may be used to distract an existing body region. In one variation, the body region is a non-soft tissue cavity. In one variation, the body region is a hard tissue cavity, such as a bone cavity arising from a tumor, injury or surgery.

FIG. 8A to 8C shows an example of inserting an implant into a bone cavity 602. In this example, the bone cavity is part of a vertebral compression fracture. Other examples of bone disorders and fractures which may be distracted include, but are not limited to, tibial plateau fractures, femoral head necrosis, osteonecrosis of the hip, knee injury, etc. FIG. 8A shows an applicator 502 inserted into a vertebral compression fracture 804 through the vertebral pedicle 808; the applicator is inserting an implant 10 into the collapsed region. The implant is shown as a linear array of pellets 12. These segments of the implant may be continuously added to the bone cavity to first fill and pack within the cavity. Once the cavity is filled, adding further segments elevates the collapsed bone. FIG. 8B shows the bone cavity after it has been distracted by application of the implant. While some of the individual segments of the implant remain joined and connected to the applicator, the user may adjust the amount of distraction by removing and/or adding segments of the implant until the shape of the collapsed vertebra has been set to an optimal shape. In one variation, the optimal shape is the natural (uncompressed) position.

Compaction of the Implant within a Cavity

Once an implant is inserted, it may be compacted within the body cavity by packing the individual segments. Any appropriate device or method may be used to compact the implant segments. These include utilizing vibration (e.g. ultrasonics, through the delivery of a second cannula or probe, for example, through the second pedicle) or physical compaction (e.g. using a curved probe or tamp through a pedicle path or with an internal or external sheath. Compaction may be particularly useful when filling hard tissue cavities such as bone cavities.

Closing a Cavity

A cavity opening through which an implant was inserted may be closed and/or sealed to maintain the compaction, and to prevent the loss of implant material from the cavity. After filling and/or distracting a cavity, a user may cut the implant and remove the applicator cannula. FIG. 8C shows that the user may also block 802 or otherwise close the opening into the bone cavity, for example, by the local application of a cement material through the cannula (or another cannula). Other methods for closing the void may include tapered pins, screws with blunt head and tip, or even screws with compressible tip members such as a spring to absorb, minimize, or prevent settling of the implant.

FIG. 9 shows an example of a screw closure 900 for use with an implant that comprises a spring 903 for applying pressure to an implant within a cavity. The screw includes threads 905. After distracting and/or filling a hard tissue cavity as described, the screw closure is screwed into the opening through which the implant was inserted. The spring-loaded tip 910 of the screw is blunt, and applies pressure onto the inserted implant. Thus, the screw can minimize any settling or further compaction that may occur after the insertion of the implant by applying pressure to help keep the implant compacted.

In general, implants and applicators as described herein may be used for filling cavities that do not require distraction.

A secondary filling material may also be used. For example, when filling a bone cavity, fluent bone filler may also be used to fill the cavity in addition to the solid implant. The combination of hard segment and fluid filler may provide added stability. The fluent material (e.g. cement) may also harden into a solid. In addition, the implant segments may reduce leakage of additional bone filler (such as bone cement) by blocking openings in the cavity that fluent filler would otherwise leak through. Less fluent filler may be needed if it is used after the solid implant, further reducing the risk of harmful leakage. In one variation, secondary filling material may be applied in conjunction with an expandable membrane around the implant segments, preventing any substantial leakage from the bone cavity.

In general, the implants and/or applicators described herein may be used to distract a cavity without being left in the cavity after distraction. For example, an implant may be used to create or enlarge a cavity. In one example, an implant may be inserted into a body region void to expand the void. The surfaces of the body region void will be compressed by the implant, causing it to expand. After removing the implant, the cavity may remain expanded, facilitating further procedures (e.g. insertion of additional devices or materials, etc). Similarly, a hard tissue cavity such as a bone cavity may be enlarged or reshaped by inserting an implant which can then be removed or left within the non-soft tissue cavity.

It may be desirable to leave the implant in the tissue for an extended period of time, up to and including the lifetime of the patient. In one variation, the implant is a permanent implant for filling and/or distracting body regions to provide long-term support and shape to the body region. In one variation, the implant is intended to be used for a period of at least six months. In one variation, the implant is intended to be used for a period of at least a year. In one variation, the implant is intended to be used for a period of many years. Implants intended for long-term use may be made of materials which do not lose a significant amount of their strength or shape over time after implantation.

Securing a Fastener

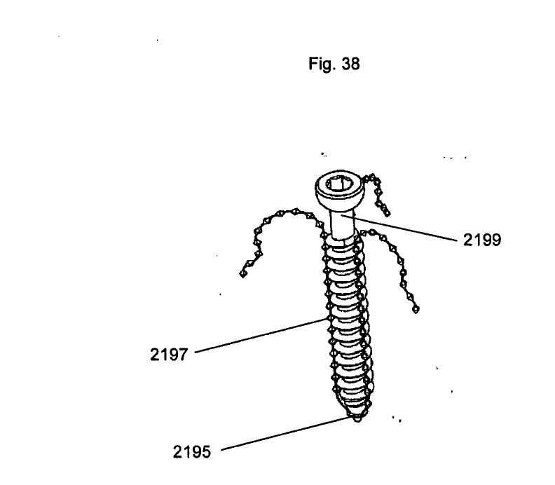

The implants and/or applicators described herein may be used to secure another implant, including fastening devices. For example, a bone screw may be inserted into an implant filling a bone cavity. Alternatively, and implant may be used to secure (or to help secure) fastening devices by coupling with the fastening device. FIG. 38 shows one variation of an implant configured to help secure a fastening device (shown as a screw). In FIG. 38, the implant is configured as an anchor that fits between the side of the fastening device and the site into which the fastening device is being inserted (e.g., a non-soft tissue such as bone). In some variations, the implant comprises a coupler (e.g., a loop, ring, hook, etc.) to couple the implant to the fastening device. Any appropriate coupler that can secure at least a portion of the implant to the fastening device may be used. The implant is coupled to the fastener so that the implant (e.g., the segments of the implant) comes between the fastening device and the site of insertion (e.g., the wall of the cavity into which the fastener is being secured).

As the fastener is secured into the body distally (e.g., into bone), the implant becomes lodged between the fastening device and the wall of the structure into which the fastener is being inserted. Thus, the implant helps anchor the fastening device. In some variations, the implant may be slightly compressible, or some of the segments may be compressible. In some variations, some of the segments (or all of them) are frangible, and may rupture under the stress of insertion to help secure the fastener into position. Some of the segments may rupture and release a bonding agent, or a catalyst to activate a fluent material or bonding agent that is included with the implant (or added to the implant), causing it to harden and further secure the fastener in position.

The implant may be connected at the distal end with several chains of segments delivered simultaneously delivered surrounding a bone screw as shown in FIG. 38. This may be particularly useful when it is desirable to use a bone screw in weakened (e.g. osteoporotic or necrotic) bone tissue. In another variation, the implant described herein may be inserted to secure an existing implant.

Hybrid Ram Applicator

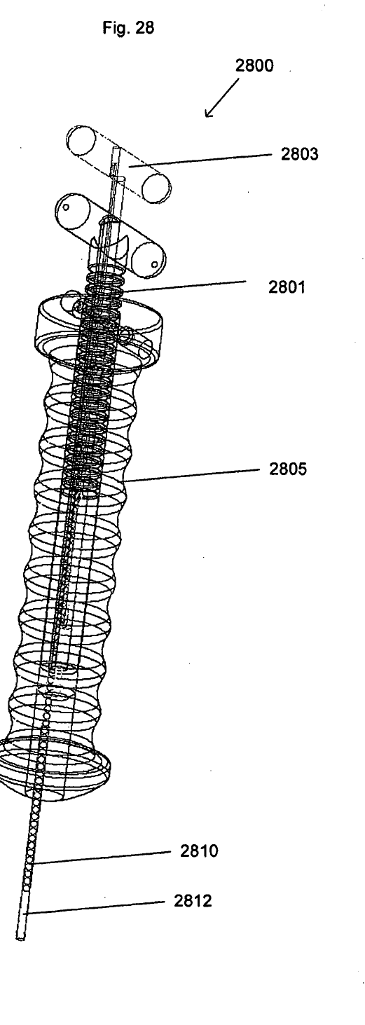

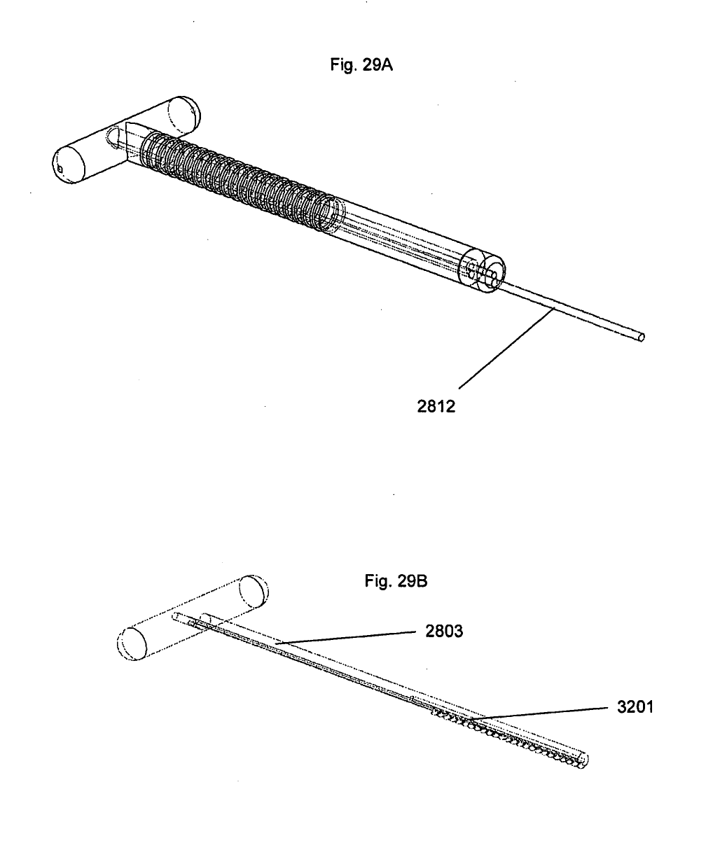

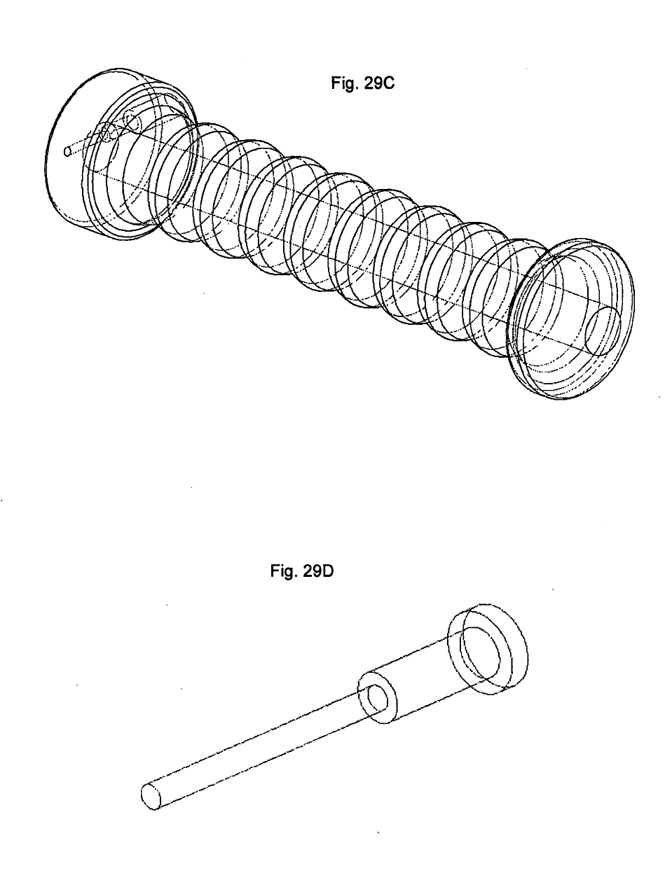

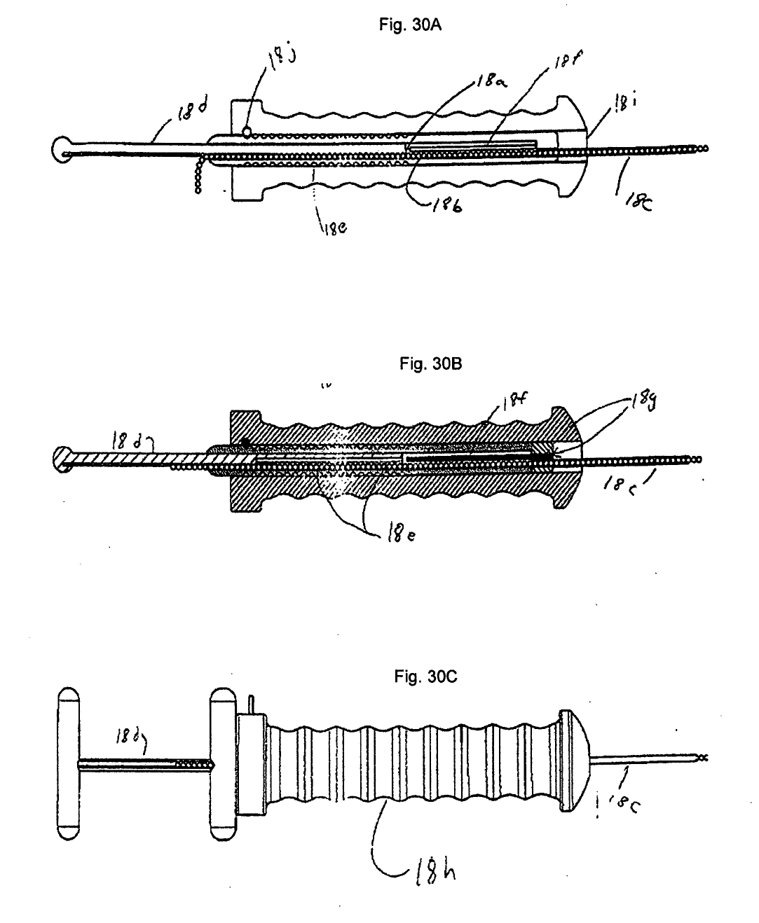

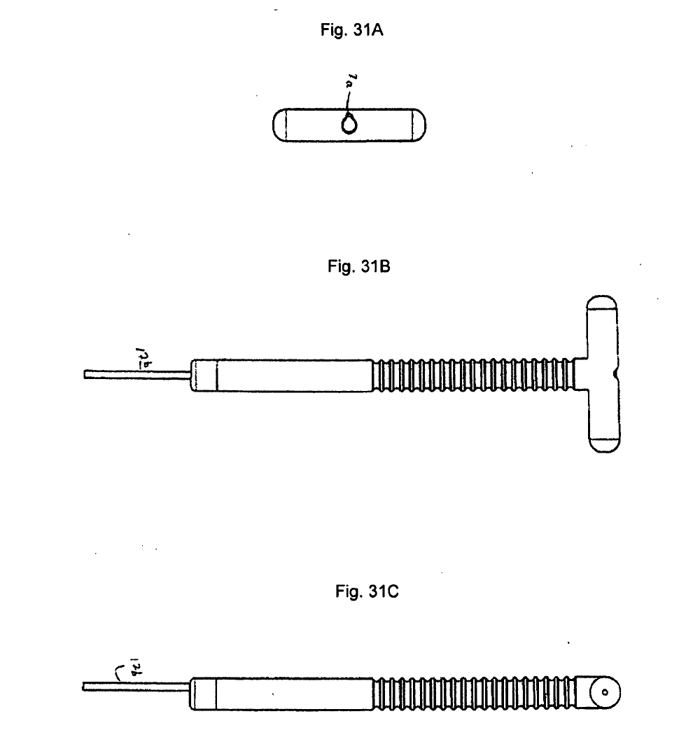

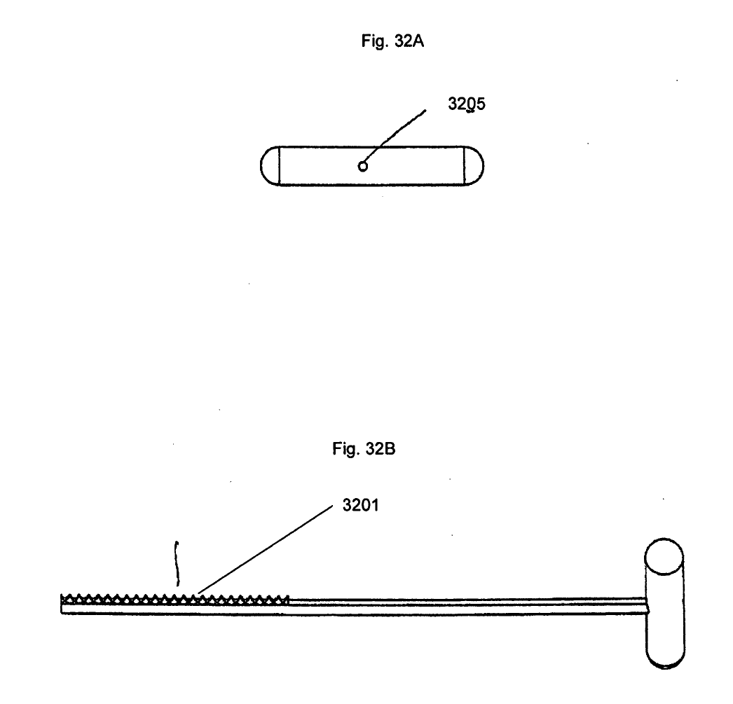

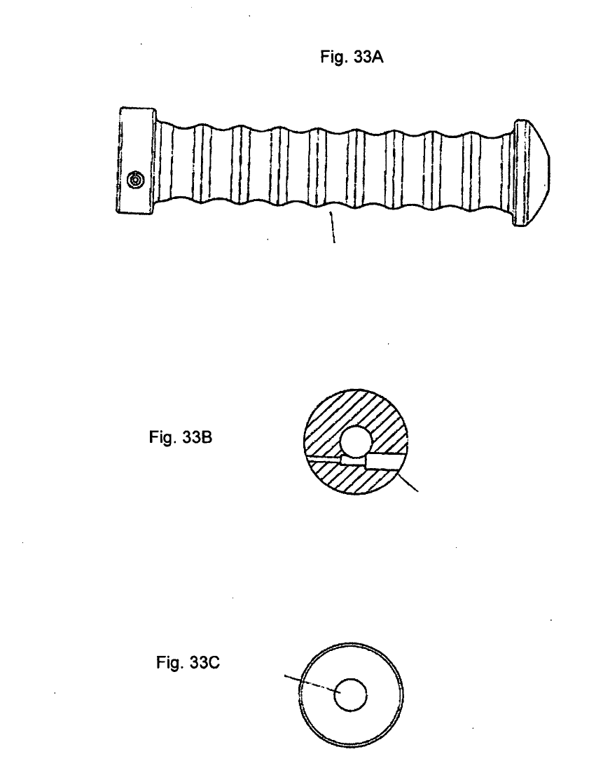

FIGS. 28 to 33 describe one variation of an applicator as described above. The hybrid ram applicator shown in FIGS. 28 to 33 combines many of the features and elements described above, and allows micro-insertion, micro-retraction, macro-insertion, and macro-retraction of some variations of the implants described above. The hybrid ram applicator may be particularly useful for applying implants into vertebral cavities, or for any cavity appropriate to receive a segmented implant as described herein.

The hybrid ram 2800 shown in FIG. 28 is composed of three primary components. First, an internal cannula 2801 (see FIGS. 29A, 30 and 31) component that is cylindrical, with two intersecting cylindrical channels running down its length. The lower channel contains a chain of implants 2810 as described above. It also contains a cannula 2812 protruding from its far end that is axially aligned with the chain of implants in the internal cannula.

Second, the hybrid ram includes a stiff member, configured as a reciprocating ram 2803 (see FIGS. 29B, and 32A-32B) that is inserted into the upper channel of the internal cannula 2801. The stiff member includes a releasable engagement region for releasably engaging at least a region of the implant. In the example shown in FIGS. 28 to 33, the stiff member is configured as a reciprocating ram that has a releasable engagement region having radial grooves 3201 (e.g., “teeth”) on at least one radial portion of the length of the reciprocating ram that can engage with the implant chain 2810 in the lower channel of the internal cannula 2801. Another radial portion of the length of the reciprocating ram includes a long axial groove 2814. When the reciprocating ram is engaged with the implant in the radial grooves 3201, by sliding the reciprocating ram along the axis of the upper channel in the internal cannula, segments of the implant can be pushed or pulled down the channel, and out (or into) the cannula at the end of the applicator, thereby inserting or retracting implant segments.

As the reciprocating ram slides forward, a cylindrical channel along the long axis of the reciprocating ram gradually mates with a guide pin 18f (shown in FIGS. 30A and 30B) that is fixed on the rotational axis of the internal cannula, which linearly aligns the implant. After sliding the reciprocating ram to its furthest extent, it can be rotated axially so that the radial grooves rotate away from the implant segments, and the long axial groove abuts the implant instead. Since the continuous axial groove does not engage (or contain) the individual segments, axial movement of the reciprocating ram does not move the implant. The axial groove only provides axial captivation of the implant chain, allowing the reciprocating ram to be retracted to its initial position without advancing or retracting the implant. The reciprocating ram can then be rotated to move the radial grooves into contact with the implant segments, so that the reciprocating ram can re-capture another length of the remaining implant chain and insert additional implants into the vertebral body. When desired elevation of the vertebral body is achieved, the reciprocating ram can once again be rotated to captivate the implant chain in the long axial groove. Once in this position, a simple ram may be inserted into the reciprocating ram through an opening in the handle 3205 (in FIG. 32A) thus allowing further manual compaction of the implants in the vertebral body.

The internal cannula is housed in a third component, an outer sheath 2805 (see FIGS. 29C and 33A) that allows for ergonomic control of the implant delivery process. The outer sheath contains a cylindrical channel along its center axis that contains the internal cannula. During the implant delivery, the depth in the vertebral body at which implant ejection occurs can be varied by translating the internal cannula along the internal void of the outer sheath. A specific depth can be maintained (e.g., in 5 mm increments) by virtue of a dual-mode locking pin (see FIGS. 29D and 33B) on the outer sheath that mates with radial grooves along the outer diameter of the internal cannula.

In summary, the described implants, applicators and methods of using them may be used to fill and/or distract a non-soft tissue including a bone cavity, in particular a vertebral compression fracture. The implant may achieve many advantages not realized with other devices intended to fill and/or distract a bone cavity. In particular, the implant described herein substantially reduces the chance of harmful leakage of bone filler material and provides three-dimensional support to the bone cavity.

Although the above examples have described primarily the filling of bone and other non-soft tissue cavities, particularly within the intervertebral body, and for treatment of vertebral compression fractures, the implants, applicators and methods described herein may be used on any tissue cavity, including but not limited to those arising from trauma, fractures, non-unions, tumors, cysts, created by a pathology or by the action of a surgeon. It will be appreciated by persons skilled in the art that numerous variations and/or modifications may be made to the described device as specifically shown here without departing from the spirit or scope of that broader disclosure. The various examples are, therefore, to be considered in all respects as illustrative and not restrictive.

Interbody Implants

Described herein are implant devices, applicators, systems and methods that may be used for interbody implants such as devices for interbody fusion, nucleus replacement, and total disc replacement. In some variations, the implants, devices and systems may be used to implant an interbody device via a relatively small cannula, allowing a small portal for insertion of the implant. In some variations, the segmented implants can be used to create an interbody fusion device that allows stable fixation within the intervetebral space. In some variations, the segmented implants and inserters may be used to create a composite nucleus replacement unit that is both elastic and stable. In some variations the segmented implants can be incorporated around a central construct thus allowing posterior delivery of an artificial total disc. Segments may interlock to form a stable, three-dimensional deployed structure (or assembled configuration). In general, a structure formed of interlocking segments is stable when the final structure cannot be readily broken apart. For example, in some structures, this means that each segment forming the structure is adjacent (and possibly interlocked with) at least two adjacent segments. The shape of the deployed structure may also provide added stability. For example, many of the deployed shapes of these implants are self-supporting, because they form ring-like structures (e.g., circular, disc, D-shapes, rings, triangular, oval, etc.) that are more stable than linear structures (such as arcs, lines, etc.) formed from component pieces. The stability of these implants is further enhanced because the deployed configuration may be larger than the opening through which they were inserted, preventing them from dislodging once implanted (e.g., within a spinal annulus). In addition, these implant may have better coverage of the spinal region (e.g., the endplates of the vertebra) into which it is implanted, providing better (e.g., more uniform) loading distribution.

A. Interbody Fusion Devices

For example, any of the segments (e.g., segmented implants), applicators and joining materials described herein may be used as part of an interbody fusion device (IBFD). In one variation, the IBFD comprises angular segments such as those shown in FIG. 11. In FIG. 11, the annular segments may be combined (e.g., implanted) to make a 360-degree annular ring, because the implant segments are curved. Furthermore, because these implants are interlocked (e.g., by interlocking regions described below), they may maintain their shape and position under a variety of stresses providing long-term stability.

FIG. 11 shows different views of one variation of a segment 1100 as described herein. As shown in the perspective view of the segment in FIG. 11A, the implant segment contains a positive male tooth 1103 on one edge (e.g., a leading or anterior edge), and a female void 1104 on an opposite edge (e.g., trailing or posterior). a portion of the female void 1104 may had a smaller diameter than the mail tooth, so that it can lock to secure the male tooth within the female void. The female void may completely surround the male tooth, or it may only surround a portion of the male tooth. FIG. 11B shows an alternative orthogonal (perspective) view of the same implant as in FIG. 11A. The segment show in FIG. 11 is perforated by several small cavities, voids or passages 1101, which may help provide stability and help allow for interstitial biological growth. Passages or cavities may be textured (e.g., roughened) or may include an anchoring region, such as a rim or lip to help attach to in-grown material or other materials (e.g., additional implants). For example, a lip region around a passage or cavity may prevent ingrown material (e.g., bone or tissue) from easily withdrawing from the implant.

FIGS. 11C, 11D and 11E provide alternate perspective views, in which the holes/passages/voids/cavities may be seen. Furthermore, each segment may comprise (e.g., be made with) an elastic or elastomeric material (including any of the materials previously described, any polymers, rubbers, gels, and the like). The segment may also contain one or more (e.g., concentric) cylindrical channels that sweep through the entire arc length of the component, as shown in FIG. 11. Thus, the passageway through the segment may comprise a curve, an angle, or any other appropriate passageway shape through which a flexible joining material (e.g., a filament) may pass. Thus, the segments may be connected (including slideably connected). In some variations, the segments are permitted only a limited slideable connection, because a stop attached to the flexible joining material may engage a region of a segment to prevent further movement of the segment along the joining material. In some variations, the segment comprises a holdfast or lock to prevent the segment from slideably moving (e.g., on the joining material) once the holdfast is engaged.

FIGS. 11F and 11G show top views and side views (respectively) of the segments shown in FIG. 11A to 11D. Sections through the segments (e.g., A to A′ and B to B′) are shown in FIGS. 11H and 11I.

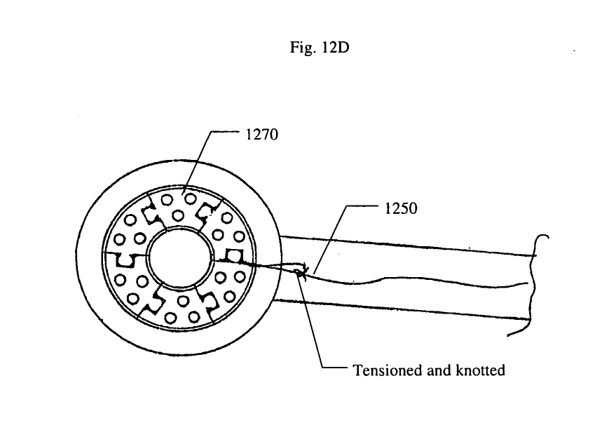

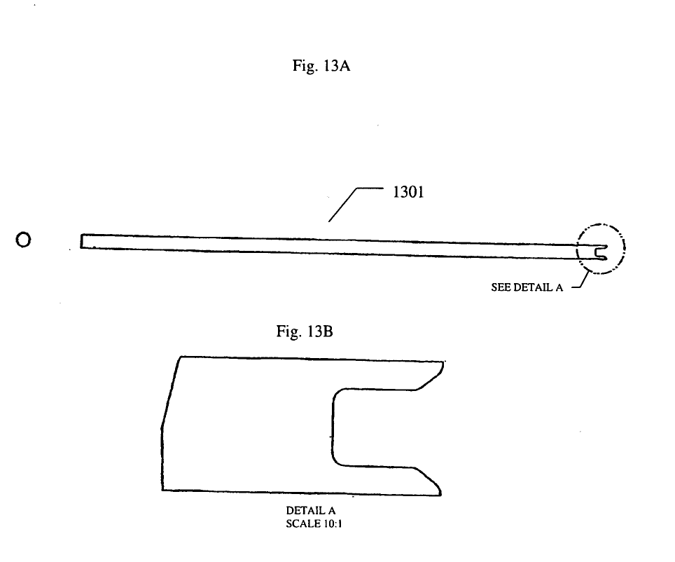

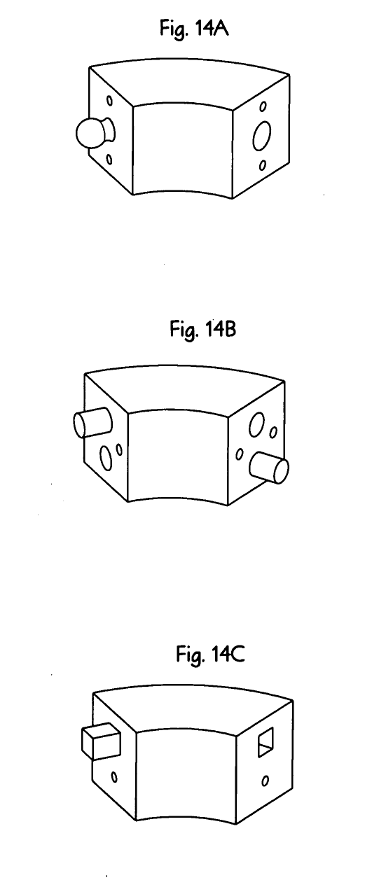

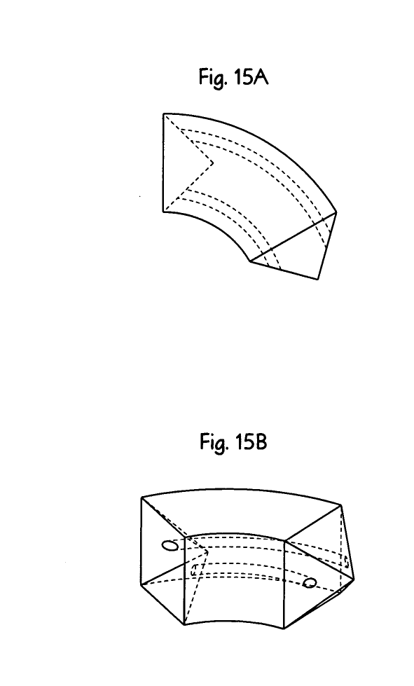

As previously described, the segments may be interlocked. For example, the male region 1103 may fit into the female region 1104 during, after, or before implantation. Thus, the segments may be interlocked to provide a final shape, such as a circle, oval, D-shape, polygon (e.g., triangle, rectangle, etc.), triangle with curving sides, etc. as described below. Thus, the segments (and therefore the flexibly connected segments and any implants comprising segments) may be thought of as an assembly comprising component parts (e.g., segments) that have the advantage of small size and maneuverability (e.g., flexibly connected), but maybe ‘reconstructed’ within the body cavity to form pre-determined shapes with properties superior to the individual components or even an unorganized collection of the individual components. For example, the compressibility, strength (e.g., crush strength), and durability of a segmented implant that has been inserted and assembled (e.g., into a ring) may be greater than even the compressibility, strength and durability of the same number of segments that have not been similarly combined. Further, the deployed shape may also have any appropriate thickness or cross-section. The ring deployed shape of FIG. 12 is shown having a uniform thickness (depth), however a variable depth (e.g., thicker in the anterior and thinner on posterior, relative to the spine) may be useful as well. Non-“flat” cross sections may also be used. For example, the upper surface, lower surface, or both, may include curves, cavities, or structures (e.g., anchor structures) to help interact with adjacent tissue structures or other implants. In variations of the implant having a central passage or opening through the deployed structures (e.g., ring-like structures), an additional segment or insert may be provided. In some variations, this insert is thinner than the deployed implant (e.g., so as not to be load bearing when inserted into the spine), and may be used, e.g., to release compounds (e.g., drugs and other therapeutics), or to interact with the implant or tissue. Another implant (e.g., configured as a disc-shaped nuclear replacement, as described above) may also be used in the center of the annular ring-type implants.

FIG. 12 illustrates one example of insertion and construction of an implant assembly. In FIG. 12, the implants are inserted into the intervetebral space in an oriented manner, so that they may be interlocked, and thus create a circular assembly (e.g., an interbody fusion). In some variations, the implant may comprise an orientation guide for maintaining (or helping to maintain) the orientation of the implants with respect to each other or with respect to the insertion site. In some variations, the orientation guide comprises a guide member. For example, a guide member may comprise a flexible member (e.g., a second flexible joining material such as a string, filament, etc., as previously described). The segments may self-orient themselves (e.g., due to interlocking, or guide regions on each segment) when tensioned (e.g., by applying tension to the flexible joining material connecting the segments). As described, tension may be used to interlock the segments. Tension may be applied (e.g., to the flexible joining material) to apply force to “snap lock” the segments together in variations that include snap-lock engagement regions. Once the segments are secured, the tension may be released. In some variations, the flexible joining material may be removed after tensioning has interlocked the segments. In some variations, tension may be permanently employed to secure the implant (e.g., by securing the flexible joining material with a knot, holdfast, or lock).

One example of an IBFD procedure is shown in FIGS. 12A to 12D. In these figures, the individual implants are similar to those shown in FIG. 11A to 11I. Of course, any appropriate implant may be used, and the final shape and size of the assembly may depend on the shape of the individual implants used. Thus, implants of different sizes may also be used in combination. For example, the implants shown in FIGS. 11 to 13 are “60° degree” implants, meaning that two of the faces (in this case the interlocking faces 1112, 1114) are angled approximately 60° degrees off of a center axis. Thus, each segment will span 60 degrees of a 360 degree circle when finally assembled. Another example may be a “45° degree” implants, and the like. The angle, shape and size of the implants (e.g., inner and outer diameters) may be customized to an individual patient's needs or anatomy. Shapes (of the total implant, as well as the individual segments) might be a circle, an oval, an ellipse, a polygon, or any variety of shapes to conform the device shape to the desired need of optimally transferring load. As described above, other shapes include ring-shapes such as circles, ovals, D-shapes, polygons of any number of sides (and the sides may be curved or linear, or both), or the like.

In FIG. 12A, the implant comprising part of the IBFD is first threaded onto one or more flexible connection(s) (e.g., flexible joining material 1204). As previously mentioned, these flexible joining materials (e.g., the filaments shown in FIG. 12A) also act as an orientation guide, keeping the segments oriented with respect to each other (e.g., so that the “top” of the segment shown in FIG. 11F may be oriented in the proper direction to correctly mate with an adjacent segment. Thus, in some variations, two flexible joining materials (e.g., flexible members, guide members, or filaments) may be used to control the orientation of the segments as they are implanted. In FIG. 12, the flexible joining material 1204 is threaded through two cylindrical channels spanning the length of the segment. The implant and flexible joining material are inserted into a cylindrical delivery cannula, 1201 and the ends of the flexible joining material 1204 are left protruding from the proximal end of the delivery cannula (not shown). Any appropriate inserter may be used to insert the segments into a body cavity (shown here as a spinal region 1205. For example, a driving probe (as shown in FIGS. 13A and 13B, and described below) may be used to insert the segments or the entire implant. FIGS. 12A to 12D show one possible progression of insertion into an intervertebral region. For example, in FIG. 12B, a second implant segment 1210 is shown inserted behind the first segment 1200. The driving probe may be configured to mate with an end of an implant (e.g., the trailing end of an implant 1200), or merely propel (e.g., push) the implant along the flexible joining material and into position.

In some variations, a guide or tether may be attached to each (or some of) the segments. When individual guides or tethers are used, a practitioner (e.g., a doctor or surgeon) may confirm placement and/or orientation of each segment via the tether, or may individually reposition or remove individual segments from the deployed implant. For example, a tether or guide may be used to remove a segment that has been interlocked with one or more other segments. As described above, the guide or tether may be stiff, flexible, or relaxed (e.g., wire or string-like material) that is attached to one or more segments. In general, the guide or tether is detachable.

FIG. 12C shows another iteration of this procedure. Another segment 1212 has been added behind the second segment 1210. Thus, subsequent implants may be moved down the flexible joining material either individually (e.g., leaving the cannula one at a time) or en masse. Similarly, once within the intervetebral region, the segments may be oriented (e.g., interlocked) as they enter, or they may arranged later during the procedure. In some variations, the segments may be “interlocked” as they enter the region. For example, the segments may comprise snap-fit components (e.g., snaps, buttons, friction-fits, etc.) to secure them into position; thus, a segment may be secured to an adjacent segment so that it will not easily move with respect to other segments or to the implantation site. In some variations, the segments comprise magnetic attachment regions. In some variations, the segments comprise mechanical attachment means (e.g., snaps, locks, etc.). In some variations, the segments are interlocked by applying tension to the flexible joining material (as previously described). Furthermore, a holdfast (e.g., a knot, a tie, a clamp, etc.) may be included as part of the implant (e.g., at one end of the flexible joining material, or between each segment) to secure one or more ends of each implant.

In any event, a final implant configuration may be achieved by inserting each segment as described herein. FIG. 12D shows a circular implant 1270. Each leading edge 1114 of each segment is shown mated to a corresponding trailing edge 1112 of an adjacent segment. Thus, the annular structure (e.g., ring) was formed by mating interlocking implant segments. An orientation guide may be used to help manipulate the segments into shape. A knot 1250 may be tied with the free ends of the flexible joining material, which may help tighten and stabilizes the composite annular implant 1270.

In some variations, interlocking the implants (for example, the implants shown in FIGS. 11 and 12) provides additional support surfaces, giving additional strength to the final implant. For example, when the interlocking members form a completed annulus as shown 1270, the assembly may withstand more forces (e.g., compressive, shear, etc.) than a similar implant comprising segments that do not interlock. Thus, segments that interlock to provide support in a final configuration may be particularly useful for treatment in body regions that undergo stresses, such as the spine.

FIGS. 13A and 13B show one variation of an inserter, configured as a driving probe 1301. The driving probe may be any driver as previously described, including a plunger-type driver, or a shaft. The driving probe may be flexible, or rigid. In some variations, the driving probe comprises an end configured to mate with one or more segments (e.g., a segment-attachable/releasable end). This end may be controllable (e.g., it may grasp and/or release a segment or part of segment). In some variations, this end may also comprise sensors to detect when a segment is attached or touching the inserter. The inserter may also comprise passageways (or channels) permitting the passage of the flexible joining material or delivery of secondary (e.g., biological or non-biological) materials around segments. In some variations, the inserter may help orient the segments (e.g., it may act as an orientation guide or as part of an orientation guide). Thus, the driving probe/inserter may allow for precise control during the insertion of each implant segment (or the entire implant) into the body (e.g., the nucleus region).

Other variations of the segmented implant may also be used, particularly other interlocking segments. For example, the segments may include a projection that inserts into another segment (e.g., a receiving portion of a segment). The segment may project into another segment so that a portion of the segment is substantially surrounded by the other segment (e.g., the insertion portion). For example, an insertion portion of a segment (e.g., a “male region”) may be surrounded by more than 180° around the insertion portion, or by more than 270°, or by 360° (completely surrounded by a portion of the second segment).

In some variations, the segments may interlock to prevent rotation and/or prevent or inhibit motion out of the plane of the implant (e.g., in the direction of the spinal axis). For example, the segments may be combined (e.g., interlocked) into an implant (e.g., by inserting, and tightening) that does not substantially flex laterally. In some variations, the segments may interlock to prevent motion of the segments relative to each other. For example, the segments may be combined (e.g., interlocked) into an implant having faces that do not rotate with respect to each other when tightened.

In some variations, the segmented implant may include a region or member that inserts into another segment and prevent removal of the segment once it is interlocked. Thus, the segment may include ribs, barbs, or the like that allow insertion but not easy removal. The segments may be interlocked permanently, or may be separable by the appropriate application of force. For example, the segments may be keyed to permit separation after they have been interlocked. A removal or separation device (e.g., an elongate member with an attachment at the distal end to fit between the segments and engage the interlocking region) may include a key to disengage the interlocking mechanism. In variations where the interlocking mechanism is a mechanical connection between the segments, the key may disengage the interlocking regions by withdrawing the connection between the segments, or by reducing the energy required to separate the segments.

FIG. 14A to 14C show perspective views of different variations of the interlocking segments as described herein. FIGS. 15A and 15B show another variation of an interlocking segment. FIG. 15A shows a top view of the segment shown in a side perspective view in FIG. 15B.

B. Nuclear Replacement

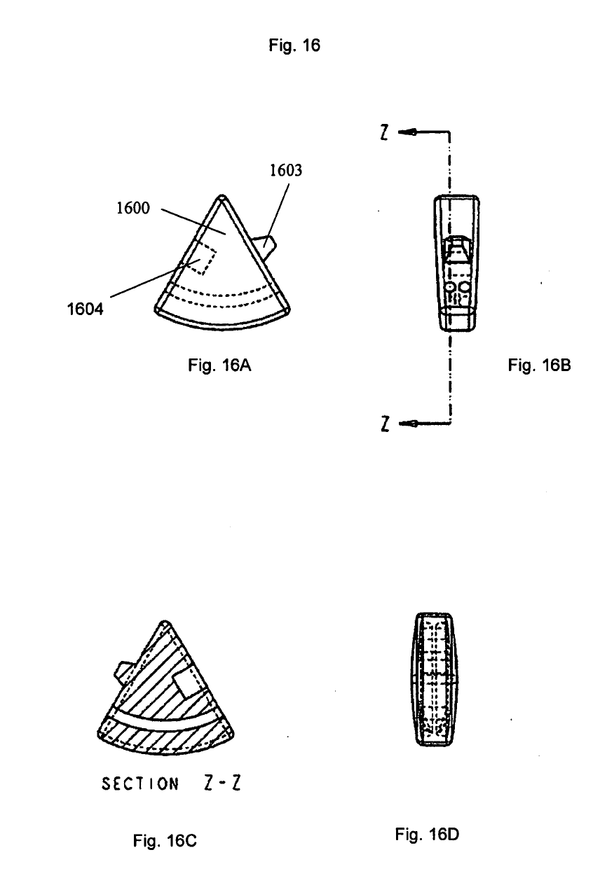

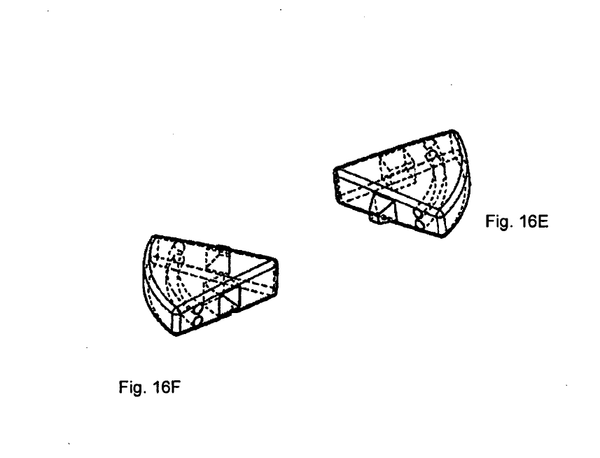

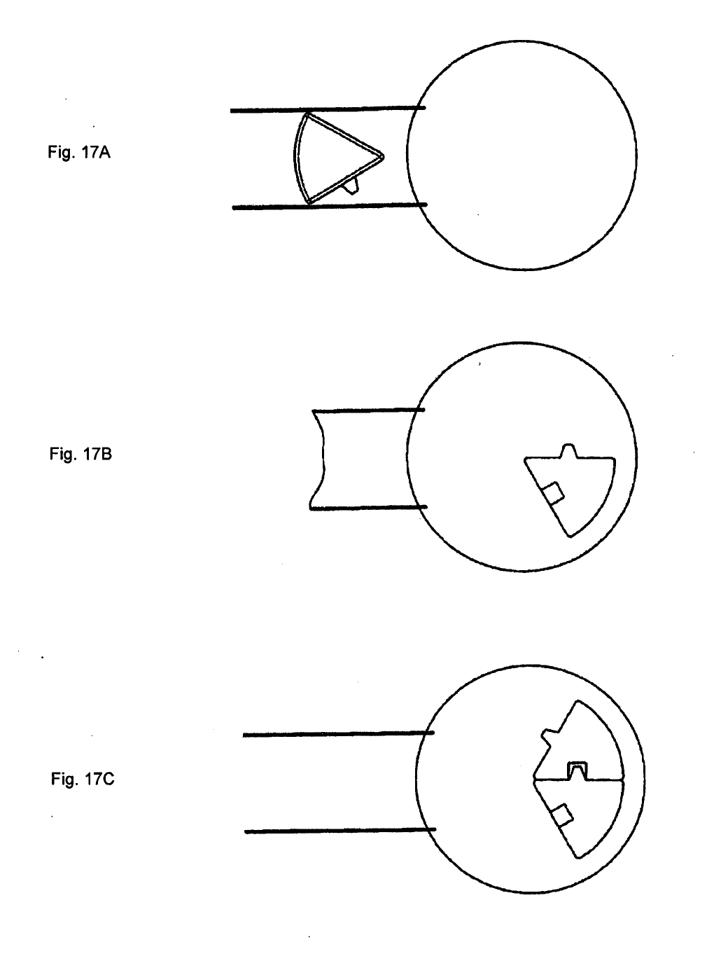

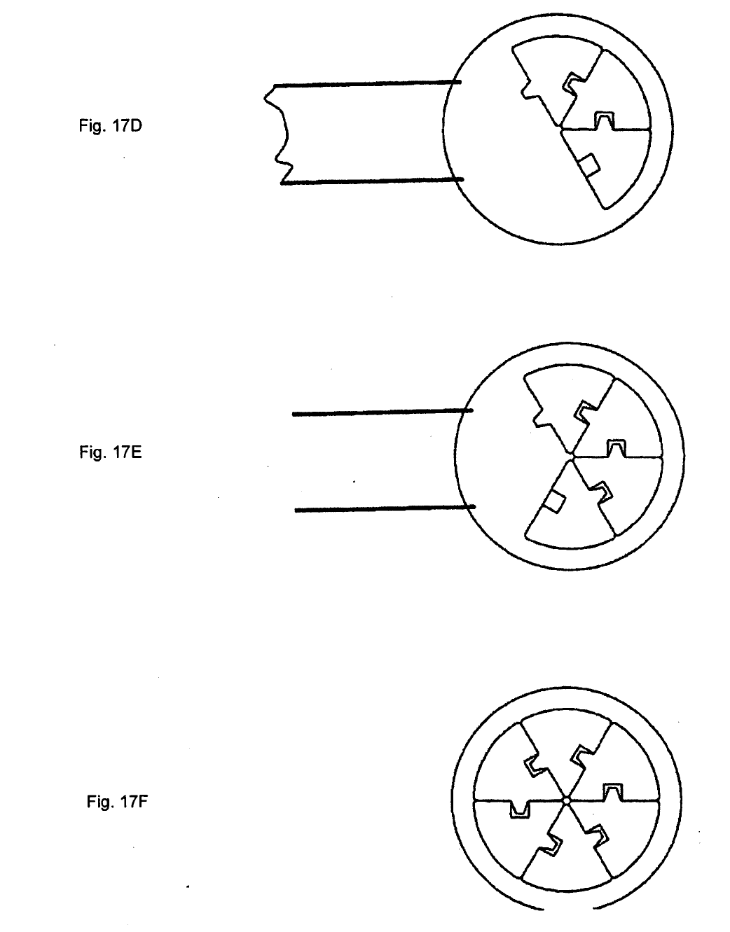

Any of the segments (e.g., segmented implants), and joining materials described herein may be used as part of a nucleus Replacement System (NRS). In one variation, the NRS comprises pie-shaped segments such as those shown in FIGS. 16A-F and 18A-C. In FIGS. 17A-F, the annular segments may be combined (e.g., implanted) to make a circular annular disk, because. In some variations, this disk is curved (e.g., has a concave or convex upper and/or lower surface). Furthermore, because these implants are interlocked (e.g., by interlocking regions described below), they may maintain their shape and position under a variety of stresses providing long-term stability. In another variation, the NRS comprises angular segments as those shown in FIG. 16.

FIGS. 16A-F shows different views of one variation of segments 1600 as described herein. As shown in the perspective view of the segment in 16A the implant segment contains at least one positive male tooth 1603 on one edge (e.g., a leading or anterior edge), and a female void 1604 on an opposite edge (e.g., trailing or posterior). FIGS. 16, 17 and 18 also provide alternative perspectives of the NRS segments and assembled implant. Furthermore, each segment of the NRS may comprise (e.g., be made with) an elastic or elastomeric material (including any of the materials previously described, any polymers, rubbers, gels, and the like). The segment may also contain one or more (e.g., concentric) cylindrical channels that sweep through the entire arc length of the component, as shown in FIGS. 16A-F. Thus, the passageway through the segment may comprise a curve, an angle, or any other appropriate passageway shape through which a flexible joining material (e.g., a filament) may pass. For example, in FIG. 16A, the segment has only a single passageway therethrough; the segment shown in FIGS. 16E and 16F has two (side-by-side) passageways therethrough. Thus, the segments may be connected (including slideably connected), and may be steered or controlled for delivery by using a connector (e.g., wire, sting, etc.) within the passageways. In some variations, the segments are permitted only a limited slideable connection, because a stop attached to the flexible joining material may engage a region of a segment to prevent further movement of the segment along the joining material. In some variations, the segments are not slideable at all.

FIGS. 16A, 16B, and 16D show top, side, and bottom views (respectively) of segments. Sections through the segments (e.g., A to A′) are shown in FIG. 16C. FIGS. 16E and 16F show perspective views of this segment.

As previously described, the segments may be interlocked. For example, the male region 1603 may fit into the female region 1604 during, after, or before implantation. Thus, the segments may be interlocked to provide a final shape, such as a circle (disk, oval, polygon, D-shape etc.), as described below. Thus, the segments (and therefore the flexibly connected segments and any implants comprising segments) may be thought of as an assembly comprising component parts (e.g., segments) that have the advantage of small size and maneuverability (e.g., flexibly connected), but maybe ‘reconstructed’ within the body cavity (formed by removal of the nucleus, for example) to form pre-determined shapes with properties superior to the individual components or even an unorganized collection of the individual components. For example, the compressibility, strength (e.g., crush strength), and durability of a segmented implant that has been inserted and assembled (e.g., into a ring or oval, bulging triangle, etc.) may be greater than even the compressibility, strength and durability of the same number of segments that have not been similarly combined. As mentioned above for the interbody fusion devices, the shape of the deployed structure may also provide added stability. For example, many of the deployed shapes of these implants are self-supporting, because they form circular or ring-like structures (e.g., disc, D-shapes, polygonal (e.g., triangular, square, etc.) shapes, oval, etc.) that are stable when the segments are interlocked because stress on the implant may be distributed evenly between the segments. The stability of these implants is further enhanced because the deployed configuration may be larger than the opening through which they were inserted, preventing them from dislodging, once implanted (e.g., within a spinal annulus).

FIG. 17A-F illustrate examples of insertion and construction of a NRS implant. In FIG. 17, the implant segments are inserted into the intervetebral space in an oriented manner, so that they may be interlocked, and thus create a circular assembly. In some variations, the implant may comprise an orientation guide for maintaining (or helping to maintain) the orientation of the implants with respect to each other or with respect to the insertion site. There may be special segments for initiating or finishing assembly of the implant. For example, the first segment may comprise special attachment sites, anchors, or guide channels (e.g., having a specific orientation), so that the rest of the implant may be assembled after (or around) it. For example, in some variations, the internal channel exits the back (e.g., the side shown in FIG. 16D) of the implant, so that when the final shape is formed, the connecting element may still be movable, and may be tensioned, tied off, or removed. Likewise, the intermediate segments and the end segment may also include specific adaptations. For example, last (or end) segment that is added to form the structure may include a holdfast region for securing (or tensioning) the connector, and the channel for the connector may exit the back, so that the connector can be readily tightened after the last segment has mated with the first segment.

In some variations, an orientation guide may be included to orient the segments so that the segments may correctly assemble into the final implant shape. An orientation guide may comprise a guide member. For example, a guide member may be a flexible member (e.g., a second flexible joining material such as a string, filament, etc., as previously described). The segments may self-orient themselves (e.g., due to interlocking, or guide regions on each segment) when tensioned (e.g., by applying tension to the flexible joining material connecting the segments).

Any appropriate implant may be used, and as will be apparent to one of skill in the art, the final shape and size of the assembly may depend on the shape of the individual implants used. Thus, implants of different sizes may also be used in combination. For example, the implants shown in FIGS. 16 to 18 are “60° degree” implants, meaning that two of the faces (in this case the interlocking faces) are angled approximately 60° degrees off of a center axis. Thus, each segment will span 60 degrees of a 360 degree circle when finally assembled into the nuclear replacement implant. Another example may be a “45° degree” implants, and the like. The angle, shape and size of the implants (e.g., inner and outer diameters) may be customized to an individual patient's needs or anatomy. The shape of the assembled nuclear replacement implant may be an appropriate shape, particularly shapes that conform to the nuclear region that was removed. For example, the top view of the shape may be circular, oval, elliptical, or any variety of shapes to conform the device shape to the desired need of optimally transferring load. Similarly the thickness and cross-sectional profile may be any appropriate size and shape. In some variation, the implant (in the deployed form) may have a lordotic curve, meaning that the implant is thicker on the anterior region and thinner on posterior portion (when implanted).

In FIG. 17A, the implant comprising part of the NRS is threaded onto one or more flexible connection(s) (e.g., flexible joining material). As previously mentioned, these flexible joining materials (e.g., the filaments shown in FIG. 12A) also act as an orientation guide, keeping the segments oriented with respect to each other (e.g., so that the segment is oriented in the proper direction to correctly mate with adjacent segments. Thus, in some variations, two flexible joining materials (e.g., flexible members, guide members, or filaments) may be used to control the orientation of the segments as they are implanted. Any appropriate applicator may be used to insert the segments into a body cavity (shown here as a nucleus region of a disk).

FIG. 17F shows an assembled NRS. Once assembled, the implant may be locked into position, so that it does not readily separate or disassemble, and so that the individual segments remain in a fixed position relative to each other. As will be evident to one of skill in the art, all of the implants described herein (including the, NRS, the IBFD and the total disc replacement) may be similarly locked into a final assembled shape. In some variations, the segments may comprise an adhesive or cement so that they may be secured. In some variations, the segments may include mechanical, electrical or magnetic fasteners. In some variations, the joining material may secure the implant into position. For example, the joining material may be tied off after tensioning to secure the implant. In some variations, the segments may be “interlocked” as they enter the region. For example, the segments may comprise snap-fit components (e.g., snaps, buttons, friction-fits, etc.) to secure them into position; thus, the segments may be secured to an adjacent segment so that it will not easily move with respect to other segments or to the implantation site. Furthermore, a holdfast (e.g., a knot, a tie, a clamp, etc.) may be included as part of the implant (e.g., at one end of the flexible joining material, or between each segment) to secure one or more ends of each implant.

In any event, a final implant configuration may be achieved by inserting each segment as described herein.

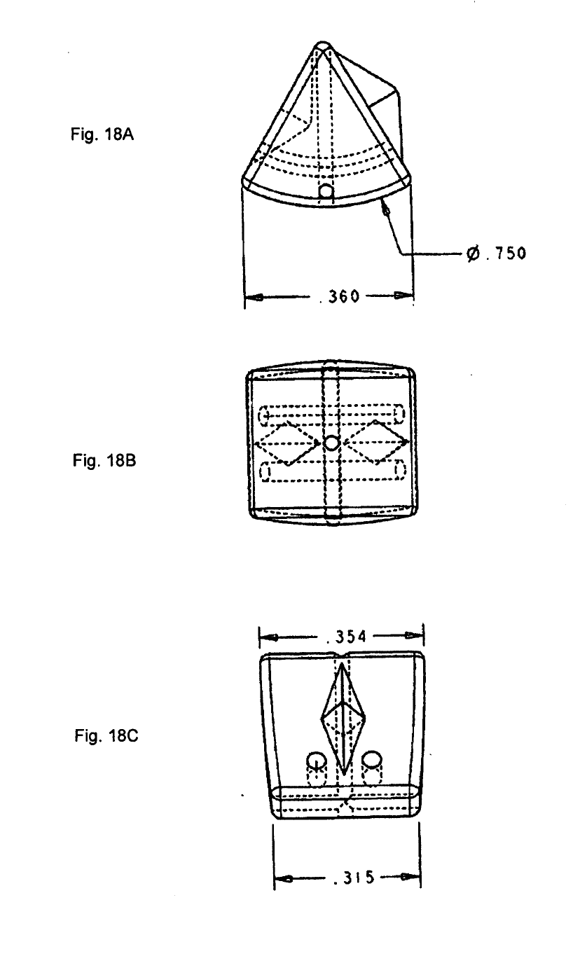

FIGS. 18A and 18B show other variations of NRS segments. Other variations of the segmented implant may also be used, particularly other interlocking segments. In some variations, the segments may interlock to prevent rotation and/or prevent or inhibit motion out of the plane of the implant (e.g., in the direction of the spinal axis). For example, the segments may be combined (e.g., interlocked) into an implant (e.g., by inserting, and tightening) that does not substantially flex laterally. In some variations, the segments may interlock to prevent motion of the segments relative to each other. For example, the segments may be combined (e.g., interlocked) into an implant having faces that do not rotate with respect to each other when tightened.

In some variations, the segmented implant may include a region or member that inserts into another segment and prevent removal of the segment once it is interlocked. Thus, the segment may include ribs, barbs, or the like that allow insertion but not easy removal.

C. Total Disc Replacement

As described above, the segmented implants described herein may include total disc replacement implants. In some variations, the total disc replacement implant includes a central organizing segment that may be jointed to a plurality of wing segments to create a replacement disc, or part of a replacement disc. In some variations, the disc replacement implant comprises an upper disc replacement component, and a lower disc replacement component. In some variations, the disc replacement implant comprises a central inner component. For example, the total disc replacement implant may comprise two articulating endplates (AEs) that may be positioned around a central inner bead (CIB).

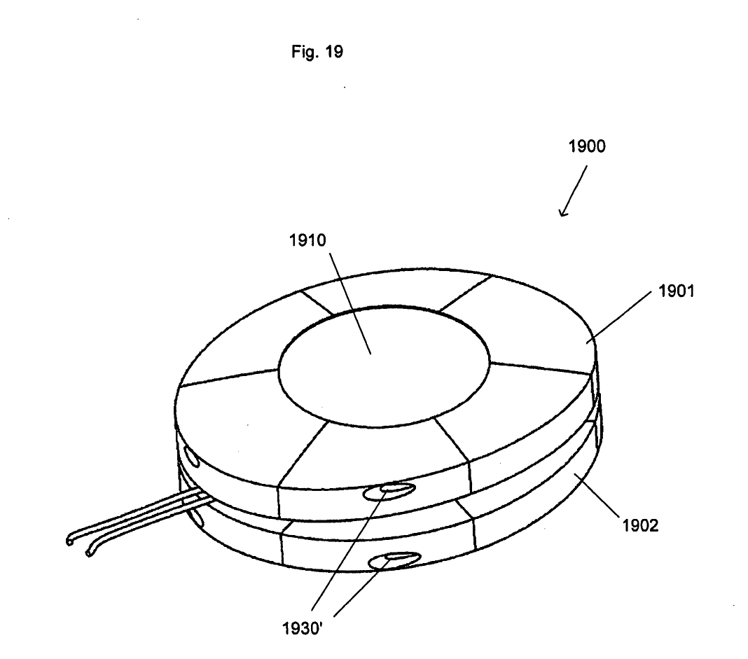

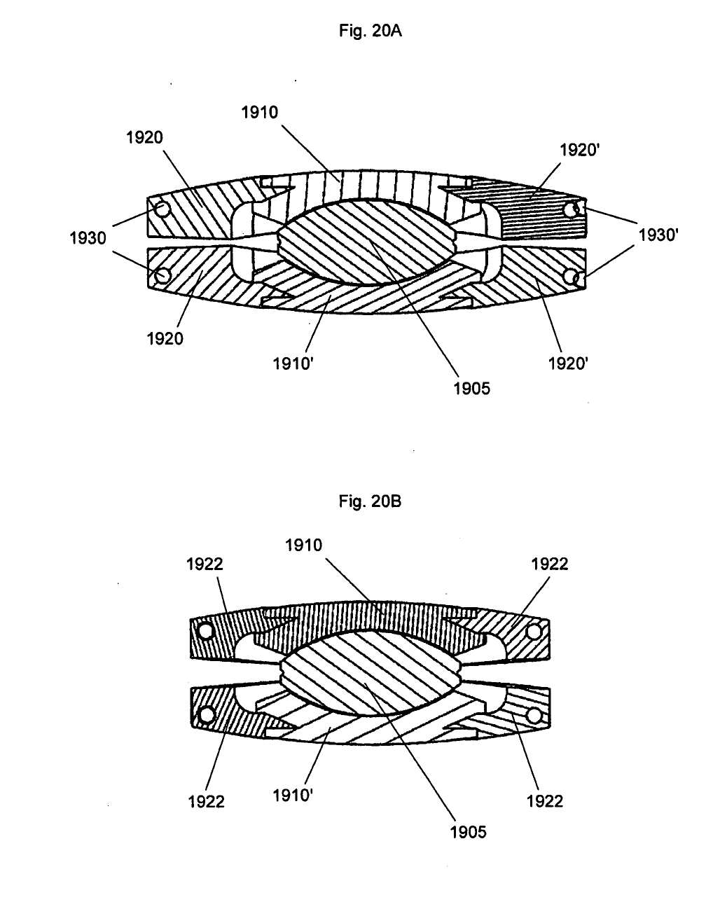

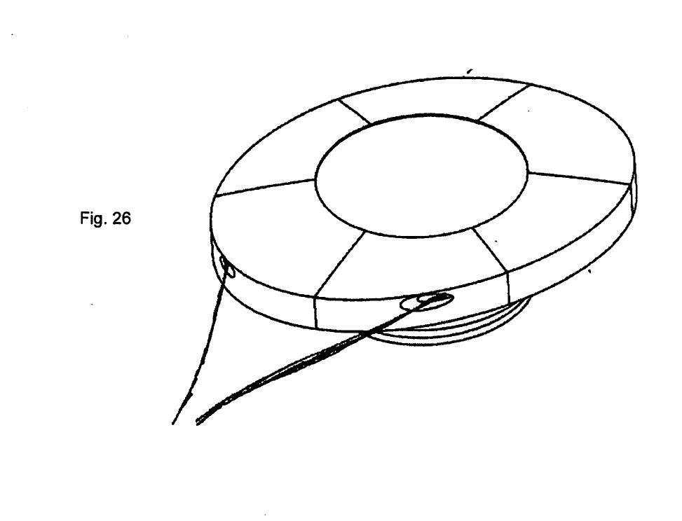

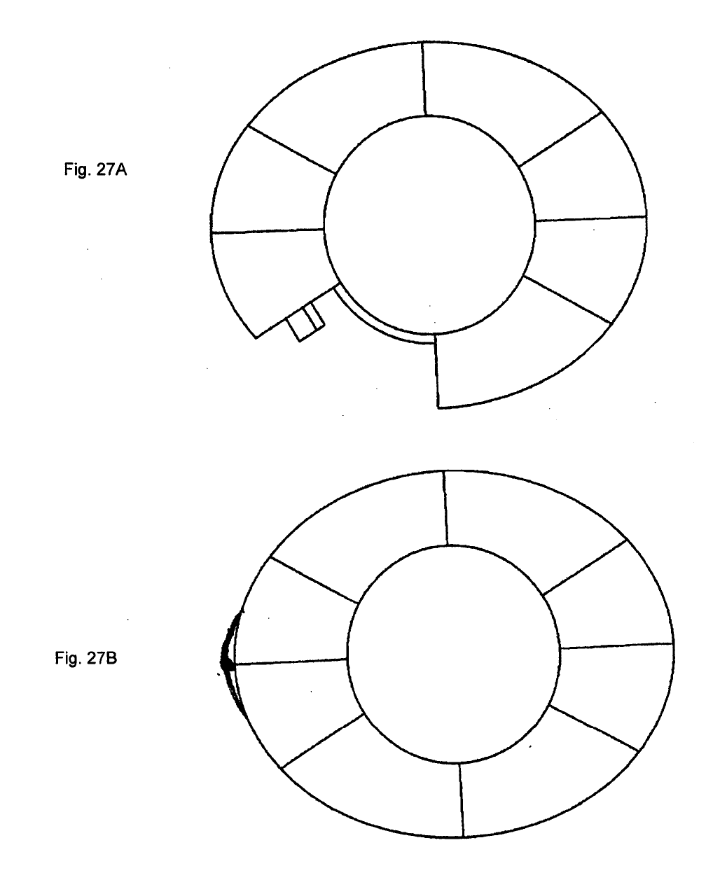

FIGS. 19-27 describe one variation of a total disc replacement implant as described herein. In general, the total disc replacement implant is a segmented implant that may be assembled into a final configuration within the spinal region space (e.g., after removing the disk region from the body). Because the segmented nature of the implant allows the device to be inserted and assembled within a cavity of the spinal region, the implant may be inserted using minimally invasive techniques. For example, the portal into which the implant is inserted may be far smaller than the portal required to insert the assembled device, leading to less trauma and a faster recovery time for a subject (e.g., a patient) receiving the device. The small segment size of the implant may also provide a surgeon with greater flexibility in how they choose to insert the device. For example, the device may be inserted posteriorly into the spine region.

The implant may be assembled around one or more central endplates, or core regions, so that the final device is stable. FIG. 19 shows an example of an assembled total disc comprising an upper AE 1901, a lower AE 1902 and an inner CIB (not visible) spacing the two. The CIB region (the central region) is shown in more detail in FIGS. 20A and 20B, which show cross-sections through a total disc replacement device similar to the one shown in FIG. 19. For example FIG. 20A shows a cross-section through the longer axis of the elliptically shaped device shown in FIG. 19. The cross-section of the CIB region is indicated 1905. The CIB region is almost totally surrounded by the upper and lower AE (the disc replacement components). Both the upper and lower AEs are constructed around a central endplate 1910, 1910′. The central endplate may provide stability both during assembly and operation of the AE. For example, the outer wing segments 1920, 1920′, 1922 may be supported by the central endplate. The two assembled AE regions may then articulate about the central region (CIB), allowing the implant to articulate, providing mobility for the spinal region into which the disc replacement has been inserted. FIGS. 19 and 20 also illustrate entrances into passages for the flexible joining material 1930, 1930′. These passages allow the segments comprising each AE to be controllably assembled around the central endplate 1910, 1910′.

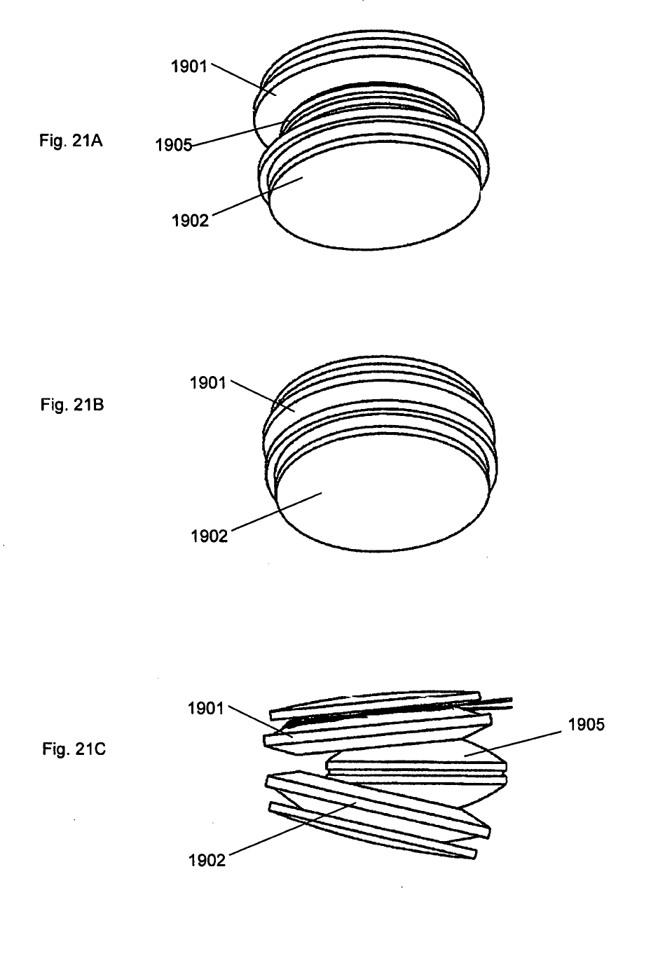

FIG. 21 shows how the upper AE, the CIB and the lower AE portions of the assembled total disc replacement implant may be arranged and interacts. FIG. 21A shows the upper AE 1901, the lower AE 1902 and the CIB 1905 region arranged sequentially. In the spinal region, the upper AE may be attached to an upper vertebral body adjacent to disc area of the spine region in which the total disc replacement is inserted. Likewise, the lower AE 1902 may be attached to a lower vertebral body across from the upper vertebral body. The CIB 1905 is located between the two AEs 1901 1902. FIG. 21C shows how the three regions may rotate with respect to each other, allowing the spine to flex when it has been inserted. The inner surfaces of the AEs are concave so that they may move against the CIB region without coming off of the CIB region.

As mentioned above, the total spinal replacement implant may be assembled from segments. The implant may be assembled in any appropriate order. For example, the upper AE, lower AE or CIB region may be implanted first. In some variations, the endplates of the upper and lower AE regions and the CIB region are inserted first, then the wing segments are added to expand the AE regions.

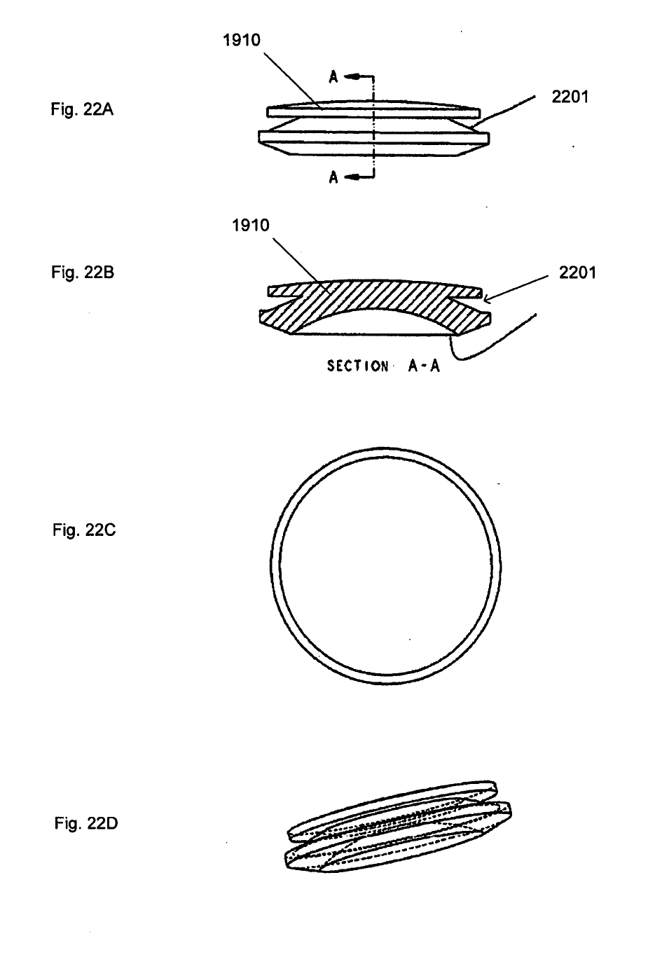

In one variation, the upper (or lower AE) central endplate region is first inserted into the cavity to hold the total disc replacement. FIGS. 22A-22D show the central endplate region 1910 for the upper AE region 1901. FIGS. 22A, 22B, 22C and 22D show side, cross-sectional, top and perspective views, respectively, of an AE central endplate. The central endplates of the AEs are designed with a depression that fits around the CIBs (e.g., the domed surface of the ICB, described below), allowing articulation of the AEs relative to the CIB. The sides of the central endplate of the AEs may have a channel 2201 (e.g., a dove tail channel or guide channel) into which the outer wing segments, described more fully below, may fit and/or lock.

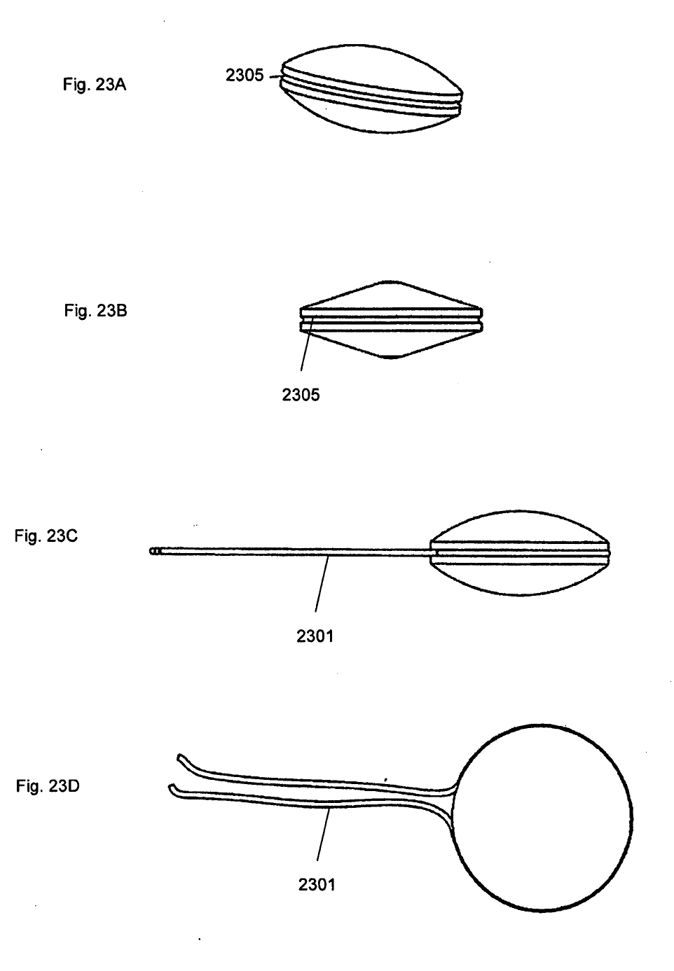

Each AE may be held in place (e.g., for insertion) using a constraining member that can secure the AEs position so that they may be held superior and inferior to the CIB. In some variations, the central endplate region of the AE is linked (e.g., connected) to a joining material along which the wing segments may be guided. The joining material may also be used to manipulate and position the central endplate and thus the AE region. Once the upper AE region is positioned, the CIB region may then be added. FIG. 23A-23D show variations of the CIB region. The CIB region may be delivered to the center of the disc space into which the implant is to be constructed, and held in place or positioned with one or more flexible constraining members. FIG. 23A shows a CIB having an oval cross-sectional profile. FIG. 23B shows a CIB having a diamond shaped cross-sectional profile. The CIB region (like the wing segments and the central endplate segments of the AEs) is simply another type of segment as described herein. FIGS. 23C and 23D show side and top profiles, respectively, of a CIB region with a flexible constraining member 2301 attached. The flexible constraining member may comprise a guiding device, or a joining member, as described herein. The CIBs illustrated in FIGS. 23A and 23B have a channel 2305 into which the constraining member 2301 may fit. The CIB may be temporarily held in place by the flexible constraining member that wraps around the CIB, and is held tight until the assembly of the total disc device is complete. Stabilization or manipulation by the constraining member can be supported with the use of tension-creating devices Stabilization or manipulation by the constraining member can be supported with the use of tension-creating devices (e.g., pinwheels, or retaining springwells). The CIB may be highly polished, or be made of (or coated with) a low-friction material. In some variations, the CIB is smooth, to prevent burnishing, galling and/or particle debris creation and contacting both of the two central cores (e.g., endplate segments), or otherwise interfering with the articulation of the implant.

The inner vertebral space may be elevated by the delivery of the segments of the total disc replacement, (e.g., the CIB and/or central endplates of the AEs), or the space may be elevated prior to insertion of the segments of the total disc replacement.

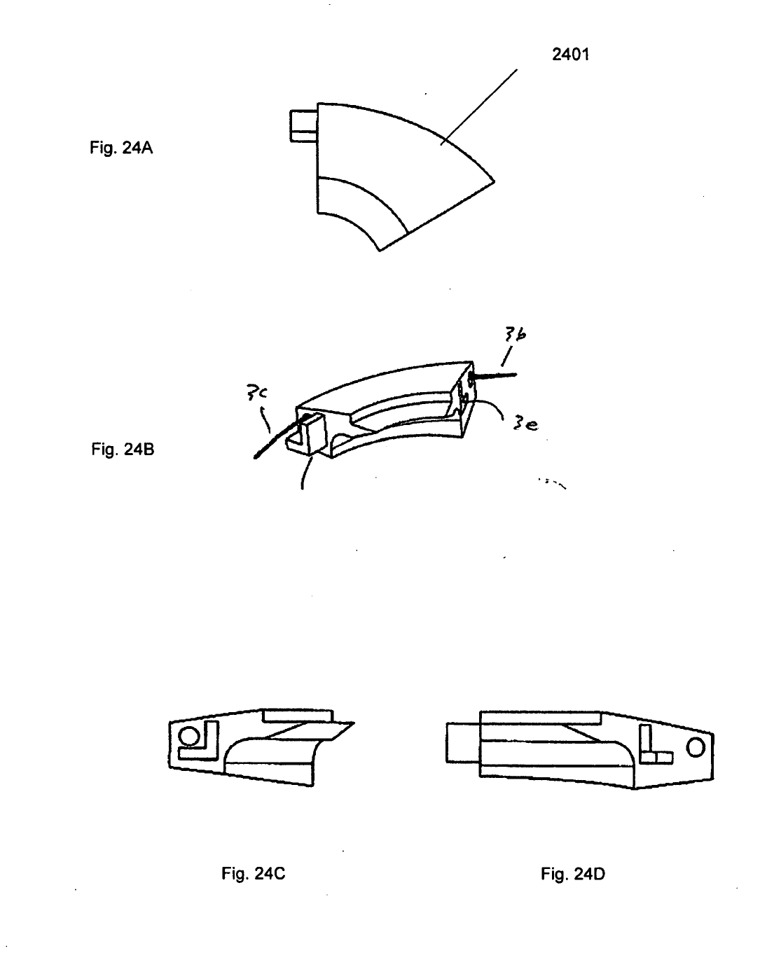

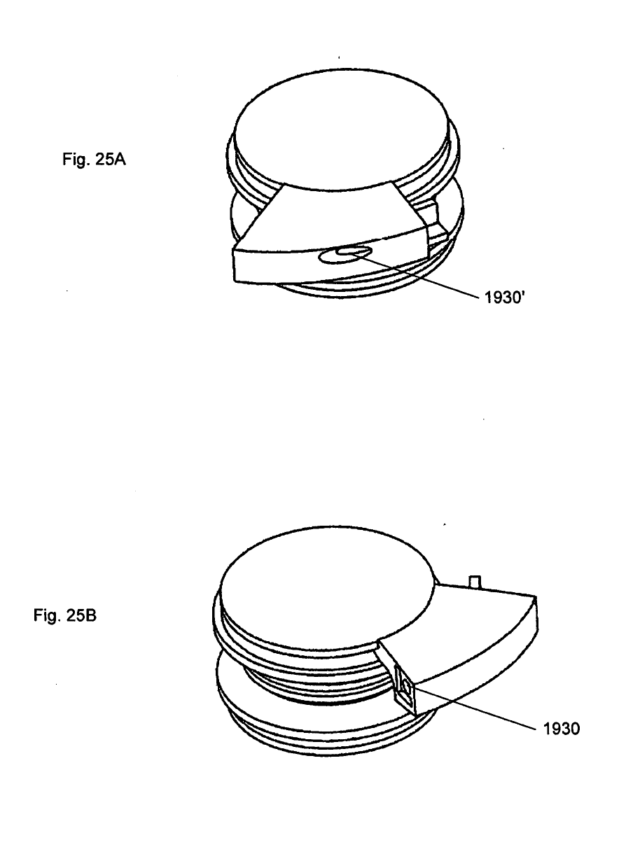

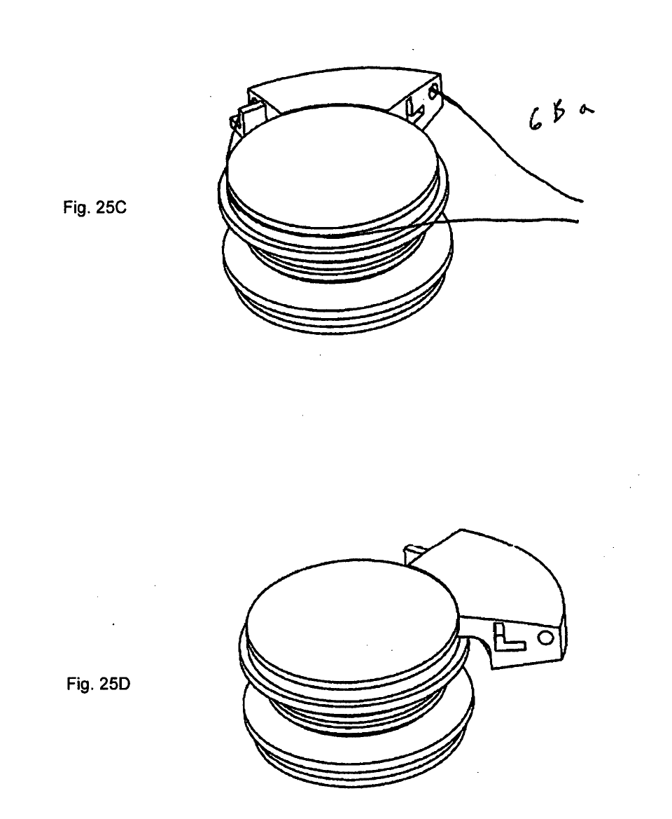

Once the central endplate region of the AEs has been inserted, the remaining segments of the AE may be added or attached to create the complete AE. FIGS. 24A to 24D show different views of locking wing segments that may be used as part of an AE, as described herein. FIG. 24A shows a top view of a wing segment. The wing segment may lock into the channel 2201 of a central endplate segment for an AE. In some variations, the wing segment is slideable within the channel of the endplate segment. As described above for the general interlocking segments and the IBFD and NRS segments, the wing segments may be sequentially placed around the central endplate, and locked into place. FIGS. 25A and 25B illustrate a central endplate of an AE in which a first wing segment has been attached. The wing segments may be connected via a joining material, as described above, and sequentially added to the central endplate. A central endplate (or a central core segment) may, in general, guide the assembly of the implant or a portion of the implant.

In some variations, 6 to 8 flexibly connected (and locking) wing segments are delivered to the AE and assembled thereon. For example, the wing segments may be delivered down the constraining member, and slid into the AE channel 2201. The locking wing segments may be connected through the insertion of a connecting member (e.g., joining material), and may lock together by any appropriate method. In some variations, the connecting member emerges from the “edge” (e.g., first and last) wing segments through a side channel 1930′, as shown. The connecting member may be tensioned, and tied off to secure the AE assembly once all of the wing segments have been added, as shown in FIG. 27B. The AE segments may be interlocked as they encircle the central endplate, so that the final assembled AE is flexibly connected and interlocked; dual locking (e.g., between the segments and around the central endplate), may create a resistance to cantilever load, creating a corresponding weight load distribution with a dual mirror plane. On both the superior (e.g., upper) and inferior (e.g., lower) AEs, the connecting member may be tightened and/or crimped, knotted or secured with a holdfast to lock the components of each AE construct into position. As described above, additional or alternative method of securing the segments into their final position may be used, including adhesives.

FIGS. 27A and 27B show top views of an AE region that is being assembled, and that is fully assembled, respectively.

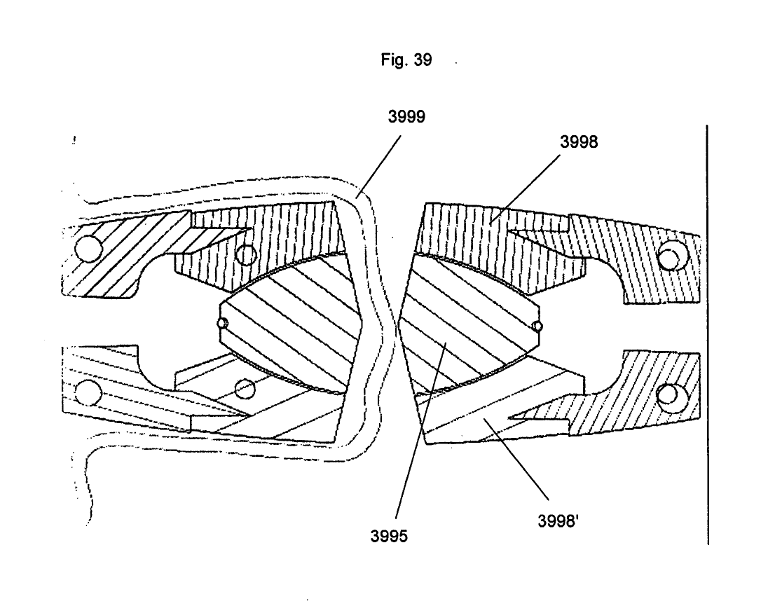

The total disc replacements described herein may also be used with additional devices, or with additional segments. FIGS. 39 to 41 provide examples of some of these additional components. In some variations, a total disc replacement system may include a centering structure around which the segments of the implant are assembled. The centering structure may be configured as a guide, and may be a flexible material (e.g., a filament such as a suture) or a stiff member, such as a pin. The other components of the total disk system may be configured to accommodate the centering feature. In operation, a centering feature may help align different implants that may otherwise move with respect to each other. When forming the total disc replacement, it may be desirable to prevent undue movement (e.g., slippage) between the implants, particularly as they are being inserted, interlocked and deployed.

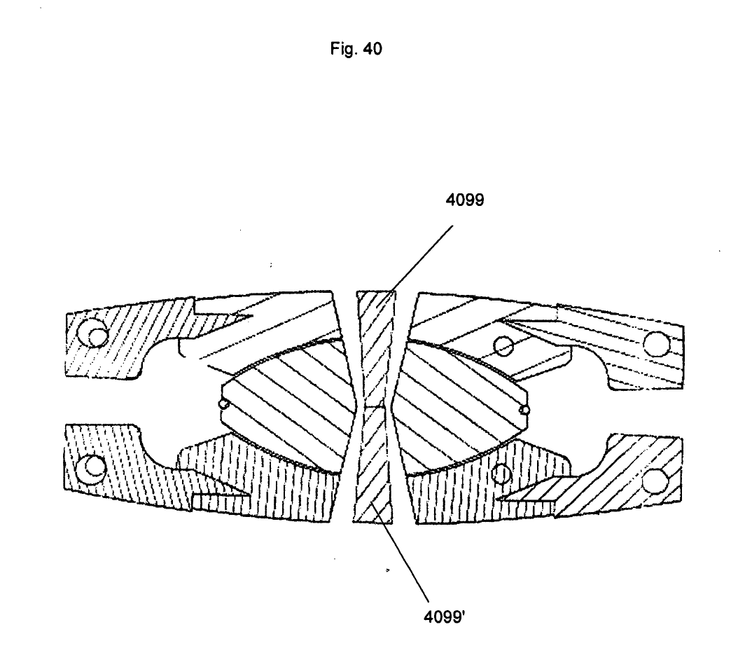

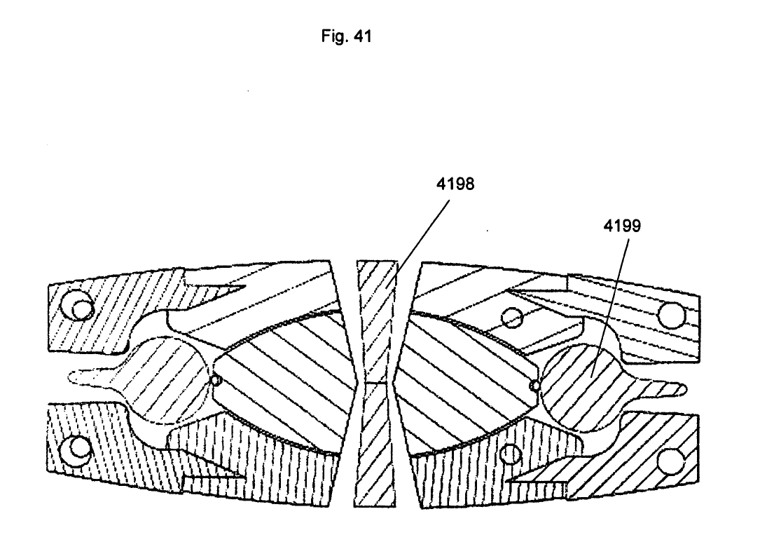

FIG. 39 shows a cross-section of a total disc replacement implant. The two articulating endplates (AEs) 3998, 3998′ are positioned around a central inner bead (CIB) 3995, and have been adapted so that a centering structure 3999 passes through all three. The centering structure may be positioned before completing assembly of the AEs and CIB (or before starting assembly of these implants), so that they can be formed around the centering structure. In some variations, the centering structure is inserted after deploying the rest of the implant. In FIG. 40, a variation of the centering structure is shown as a pin 4099. The stiff pin may comprise two or more segments 4099, 4099′ that themselves interlock. The centering structure may be removable. For example, when an interlocking pin is used, the centering structure may be removed by disengaging the interlocking pins, and removing the segments. In some variations, the centering feature remains within the implant, acting as a limiter on the movement, limiting the amount that the different components of the implant may move relative to one another. In any event, the centering feature may be “locked” within the implant, as shown for the pin-type 4099, 4099′ centering structure in FIG. 40, which has flanged ends preventing the pin from being easily removed from the implant. The upper and lower portions 4099, 4099′ of the pin may be disengaged, unlocking the pin. As shown in FIGS. 39 and 40, the region of the implant into which the centering structure fits may be larger than the centering structure, permitting a limited range of motion.

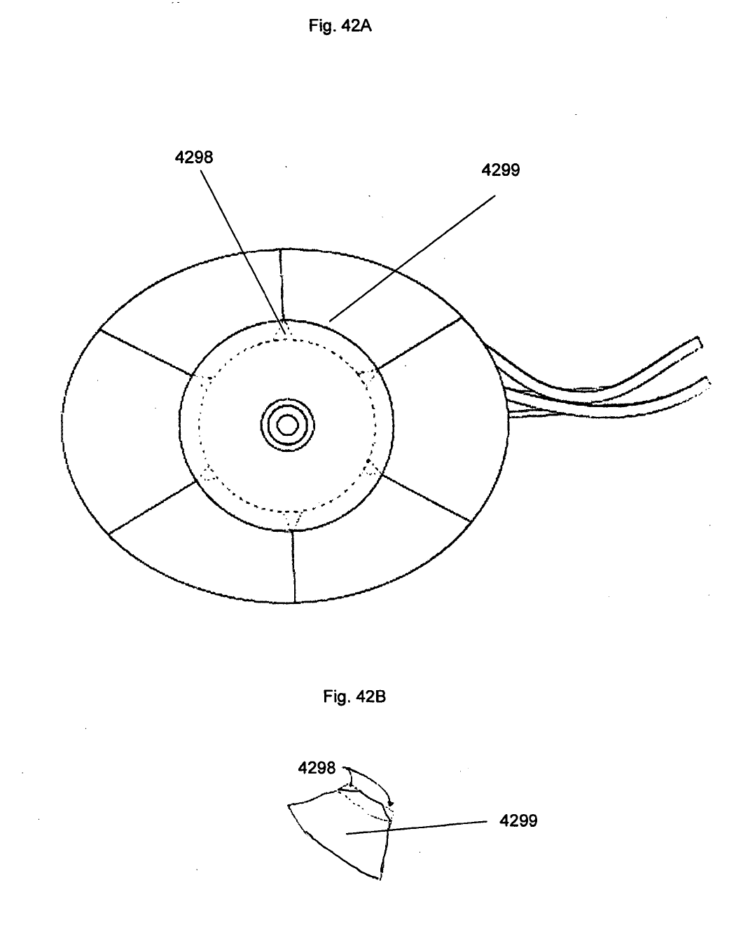

FIG. 41 shows a total disc replacement assembly which includes a circumferential compliance ring 4199, around which the implant has been assembled. This compliance ring may also help limit motion between the different components of the total disc replacement and/or the surrounding tissue. For example, the compliance ring may comprise a flexible (e.g., elastomeric, foam, etc.) material which is inserted into the body before completing deployment of the different components of the implant (e.g., the AEs and the CIB). In one variation, the total disc replacement system is assembled by first preparing the spinal region where the implant will be inserted (e.g., the intravertebral space). Preparation may involve removal of tissue, distraction (e.g., widening the region of the body where the implant will be inserted) and pre-treatment with medicaments (e.g., biologics such as BMP, etc.). In some variations, the tissue is prepared by forming the cavity as described herein. Since the implants are delivered as discrete segments, the spinal region may be accessed through a small (e.g., smaller than the deployed implant) opening made from the dorsal side of the subject (posterior delivery). A tissue distracter may be inserted into the same opening to expand the cavity into which the implant will be received (for example a manual forceps spinal distracter may be used). Once the cavity is prepared, the segments of the implant may be inserted and deployed.

In the variation shown in FIG. 41, the centering structure 4198 (attached to a tether or guide, not shown) is inserted, and one of the AEs is inserted as described above. For example, the central endplate may be inserted and positioned so that the centering structure is with a central opening. The wing segments of the AE may then be inserted and interlocked to form the AE within the cavity (in some variations, the wing segments may be added later). A compliance ring 4199 may then be inserted and positioned with respect to the inner side of the AE. Since the ring may be flexible, it may be collapsed or compressed in order to fit within the space. Next the CIB is inserted. In some variations, the CIB is threaded over the guide or tether that remains attached to the centering structure (in variations where the CIB comprises segments, they may be inserted and interlocked around the centering structure). The second AE may then be assembled by inserting the central endplate of the second AE (e.g., threading it over the guide) and then assembling the wing segments of the second AE and interlocking them into position. As with all of these devices, many of the techniques known in the art for minimally invasive procedures may be used to guide, position and secure the segments into position. Guides or tethers may be attached to part of the implant (e.g., the central endplate) or anchored within the body to help position and assembly the deployed structure. After inserting and assembling the implant, the tissue distraction (e.g., caused by a tissue distracter) may be removed.

Although the examples provided herein describe certain variations of the order that segments may be inserted and assembled within the body, many different variations are possible. For example, the centering feature may be added after assembling the rest of the implant assembly.

FIG. 42 illustrates another variation of an AE in which the segments have been adapted by including passages or regions for collecting debris. As described above, the implants may be positioned within a prepared spinal region. However, debris (e.g., tissue debris) may become lodged between segments or on the segments, inhibiting the ability of the segments to interlock properly. This may be particularly problematic when sliding the segments into position. In FIG. 42A, the individual wing segments 4299 include a cut-out region 4298 forming a relief section when the segments are joined. Similar relief sections may be included in any appropriate position of the segments. Detail of an individual wing segment 4299 is shown in FIG. 42B.

In summary, the described implants, applicators and methods of using them may be used to fill and/or distract a non-soft tissue including a bone cavity, in particular degenerative disc disease. The implant may achieve many advantages not realized with other devices intended to fill and/or distract a bone cavity. In particular, the implants described herein substantially improves containment of interbody implants because of a small entry portal and otherwise intact annulus. The implants may also provide greater coverage of bony regions, providing an improved loading pattern, and may therefore improve support to the bone cavity.

Any of the features described in each of the sections provided above (e.g., the interbody fusion devices, total disc replacement, or nuclear replacement) may be used with any of the variations and embodiments of the devices, systems and methods described herein.

Although the above examples have described primarily the filling of bone and other non-soft tissue cavities, particularly within either the intervertebral body for treatment of degenerative disc disease or the intravetebral body for treatment of vertebral compression fractures, the implants, applicators and methods described herein may be used on any tissue cavity, including but not limited to those arising from trauma, fractures, non-unions, tumors, cysts, created by a pathology or by the action of a surgeon. It will be appreciated by persons skilled in the art that numerous variations and/or modifications may be made to the described device as specifically shown here without departing from the spirit or scope of that broader disclosure. The various examples are, therefore, to be considered in all respects as illustrative and not restrictive.

We claim:

1. An implant for insertion into the spinal region of a subject, comprising:
a plurality of interlockable segments, deployable from a delivery configuration into a deployed configuration;
wherein, when the implant is in the delivery configuration, the implant comprises a linear array of the segments that are flexibly connected, and when the implant is in the deployed configuration, the segments are interlocked into a stable structure so that each segment is adjacent to and interlocked with at least two other segments.

2. The implant of claim 1, further comprising a filament connecting the segments.

3. The implant of claim 2, wherein at least some of the segments are slideably coupled to the filament.

4. The implant of claim 1, wherein the segments interlock by mating with adjacent segments in the deployed configuration.

5. The implant of claim 1, further comprising a holdfast to secure the implant in the deployed configuration.

6. The implant of claim 1, wherein the deployed configuration is configured as a ring.

7. The implant of claim 1, wherein the deployed configuration is configured as a disc.

8. A method of inserting an interlockable implant comprising:
positioning an applicator adjacent to a target tissue site;
delivering an implant to the target tissue site, wherein the implant comprises a linear array of flexibly connected interlockable segments; and
securing the implant into a deployed configuration wherein the interlockable segments are interlocked so that each segment is adjacent to and interlocked with two other segments.

9. The method of claim 8, further comprising deploying the implant from a delivery configuration in which the implant comprises a linear array of flexibly connected segments to a deployed configuration in which the segments are interlocked into a stable structure so that the interlocked segments do not move with respect to adjacent segments.

10. The method of claim 9, wherein the step of deploying comprises tensioning the connection between the segments.

11. An interbody fusion device for insertion into the spinal region of a subject, comprising:
a plurality of interlockable segments, deployable from a delivery configuration into a deployed configuration;
wherein, when the implant is in the delivery configuration, the implant comprises a linear array of the segments that are flexibly connected, and when the implant is in the deployed configuration, the segments are interlocked into a ring wherein each segment is adjacent to and interlocked with at least two other segments.

12. The interbody fusion device of claim 11, wherein at least some of the segments comprise voids configured to allow ingrowth.

13. The interbody fusion device of claim 11, wherein the segments are slideably coupled to a filament passing though one or more passages within each segment.

14. The interbody fusion device of claim 11 further comprising an orientation guide configured to maintain the orientation of the segments with respect to each other in the delivery configuration.

15. The interbody fusion device of claim 11, wherein each segment comprises two faces offset by between about 30 and about 60 degrees, and wherein each face is configured to interlock with an adjacent face of a another segment.

16. A method of inserting an interbody fusion device comprising:
positioning an applicator adjacent to a target tissue site;
delivering an interbody fusion device to the target tissue site, wherein the device comprises a linear array of flexibly connected interlockable segments; and
securing the device into a deployed configuration wherein the interlockable segments are interlocked so that each segment is adjacent to and interlocked with two other segments to form a ring.

17. An nuclear replacement device for insertion into a subject's spine, comprising:
a plurality of pie-shaped interlockable segments, deployable from a delivery configuration into a deployed configuration;
wherein, when the implant is in the delivery configuration, the implant comprises a linear array of the segments that are flexibly connected, and when the implant is in the deployed configuration, the segments are interlocked into a disc wherein each segment is adjacent to and interlocked with at least two other segments.

18. The nuclear replacement device of claim 17, wherein at least a region of the segments comprises an elastic material.

19. The nuclear replacement device of claim 17, wherein the segments are slideably coupled to a filament passing though one or more passages within each segment.

20. The nuclear replacement device of claim 17, further comprising an orientation guide configured to maintain the orientation of the segments with respect to each other in the delivery configuration.

21. The nuclear replacement device of claim 17, wherein each segment comprises two faces offset by between about 30 and about 60 degrees, and wherein each face is configured to interlock with an adjacent face of a another segment.

22. A method of inserting a nuclear replacement device comprising:
positioning an applicator adjacent to a target tissue site;
delivering a nuclear replacement device to the target tissue site, wherein the device comprises a linear array of flexibly connected interlockable segments; and
securing the device into a deployed configuration wherein the interlockable segments are interlocked so that each segment is adjacent to and interlocked with two other segments to form a disc.

23. A total disc replacement device for insertion into a subject's spine, comprising:
a plurality of interconnecting segments, deployable from a delivery configuration into a deployed configuration;
wherein the segments comprise:
a central endplate; and
a plurality of wing segments;
further wherein, when the device is in the delivery configuration, the device comprises a central endplate and a linear array of the wing segments that are flexibly connected, and when the device is in the deployed configuration, the segments are interlocked around the central endplate into an articulating endplate.

24. The total disc replacement device of claim 23, further comprising a central inner bead configured to abut at least the central endplate region of the articulating endplate.

25. The total disc replacement device of claim 23, further comprising a second plurality of interconnecting segments, deployable from a delivery configuration into a deployed configuration;
wherein the second plurality of segments of comprise:
a second central endplate; and
a second plurality of wing segments;
further wherein, when the device is in the delivery configuration, the device comprises a second central endplate and a linear array of the second wing segments that are flexibly connected, and when the device is in the deployed configuration, the second plurality of segments are interlocked around the second central endplate into a second articulating endplate.

26. The total disc replacement device of claim 25, wherein the central inner bead is further configured to abut a second articulating endplate.

27. The total disc replacement device of claim 25, wherein the central endplate comprises a disc having a channel for mating with the wing segments.

28. The total disc replacement device of claim 25, wherein at least a region of the central endplate comprises a smooth surface for mating with a central inner bead.

29. The total disc replacement device of claim 25, wherein the annular endplate comprises a concave surface for coupling with the central inner bead.

30. The total disc replacement device of claim 25, wherein the segments are slideably coupled to a filament passing though one or more passages within each segment.

31. The total disc replacement device of claim 25, further comprising an orientation guide configured to maintain the orientation of the segments with respect to each other in the delivery configuration.

32. The total disc replacement device of claim 25, wherein the orientation guide comprise a filament connecting the segments.

33. A method of inserting a total disc replacement device comprising:
positioning an applicator adjacent to a target tissue site;
delivering an articulating endplate to the target tissue site, wherein the articulating endplate comprises a linear array of flexibly connected interlockable segments comprising a plurality of wing segments for mating with a central endplate; and
securing the articulating endplate into a deployed configuration wherein the wing segments and the central endplate are interlocked.

34. The method of claim 33, further comprising delivering a central inner bead to the target tissue site.

35. The method of claim 34, further comprising delivery a second articulating endplate to the target tissue site, wherein the second articulating endplate comprises a linear array of flexibly connected interlockable segments comprising a plurality of wing segments for mating with a central endplate.
